(19) United States
(12) Patent Application Publication
(10) Pub. No.: US 2009/0248141 A1
(43) Pub. Date: Oct. 1, 2009

(54) Shape Memory Polymer Medical Devices

(75) Inventors: Robin Shandas, Boulder, CO (US); Christopher M. Yakacki, Atlanta, GA (US); Kenneth Gall, Atlanta, GA (US); Alex E. Eckstein, Centennial, CO (US); Michael Lyons, Boulder, CO (US); Devatha P. Nair, Lakewood, CO (US)

Correspondence Address: HENSLEY KIM & HOLZER, LLC, 1660 LINCOLN STREET, SUITE 3000, DENVER, CO, 80264, US

(73) Assignee: THE REGENTS OF THE UNIVERSITY OF COLORADO, Denver, CO (US)

(21) Appl. No.: 12/295,594

(22) PCT Filed: Mar. 30, 2007
(86) PCT No.: PCT/US07/65691

§ 371 Date: Sep. 30, 2008

Related U.S. Application Data
(60) Provisional application No. 60/788,540, filed on Mar. 30, 2006.

(51) Int. Cl. A61F 2/92; A61M 29/00; A61M 29/04; B29C 35/02

(52) U.S. Cl. 623/1.19; 623/1.18; 606/198; 606/194; 264/494

(57) ABSTRACT

Medical devices for in vivo medical applications are disclosed. The medical devices are constructed of shape memory polymer (SMP) materials capable of assuming a memory shape at physiological temperatures. These medical devices may be used in surgical procedures and in both vascular and non-vascular applications. These SMP medical devices have a post-implantation memory shape that is substantially identical to or slightly larger than the insertion site to adapt to vessel growth or size changes. SMP medical devices may be formed as stents or occlusion devices (i.e., plugs) having a number of different structural features. The SMP medical devices may be formed from a first monomer and a second cross-linking monomer, wherein the weight percentages of the first and second monomers are selected by performing an iterative function to reach a predetermined glass transition temperature (T_{g}) and a predetermined rubbery modulus to optimize post-implantation memory shape properties of the devices.

### CROSS-REFERENCE TO RELATED APPLICATION

The present application claims the benefit of and priority to the prior-filed U.S. Provisional Patent Application, No. 60/788,540, filed Mar. 30, 2006, entitled “Shape Memory Polymer Medical Device,” the subject matter of which is hereby specifically incorporated herein by reference for all that it discloses and teaches.

### STATEMENT REGARDING FEDERALLY SPONSORED RESEARCH OR DEVELOPMENT

This technology was developed with sponsorship by the National Institute of Health Grant No. EB004481-01A1 and Grant No. HL067393 and the government has certain rights to this technology.

### BACKGROUND DESCRIPTION OF THE RELATED ART

Cardiovascular disease (CVD), principally heart disease and stroke, is the leading cause of death for both men and women in the US. Almost one million Americans die of CVD each year, accounting for 42% of all deaths. A significant portion of these deaths are caused by coronary artery disease, the clogging of the arteries by cholesterol buildup. Current treatment includes balloon angioplasty coupled with stenting. Presently, most FDA approved stents consist of stainless steel framework or NiTi shape memory alloys.

There are several major problems with the use and implantation of the current stents (both cardiovascular and non-cardiovascular, i.e., urologic, biliary, esophageal, gynecological and pulmonary) including invasiveness of the procedure, inflammatory or other non-ideal biological host response, mismatch between the mechanical properties of the stent with those of the host vessel, limited ability to deliver drugs over long periods of time, and limited thermomechanical response (i.e., fast deployment that produces arterial wall damage). Drugs are attached to polymer coatings on current metal stents using a variety of methods (e.g., bonding, suspension, etc.). However, the extremely thin layer of polymer coatings for these stents inherently limits the amount of drugs that can be present.

Several congenital and acquired diseases produce unwanted connections within blood vessels that decrease the efficiency of the cardiovascular or non-vascular system. Such types of connections include patent ductus arteriosus (PDA), aorto-pulmonary shunts, unwanted collateral vessels, and arterio-venous shunts. Non-vascular connections include fistulas such as broncho-pleural fistulas, entero-cutaneous fistulas and pancrea-cutaneous fistulas. These connections require surgical or interventional closure. Current interventional closure methods typically use metal (e.g., stainless steel or a NiTi shape memory alloy, such as Nitinol® for example) devices that are delivered via a catheter and expanded to block the lumen. Due to the limited expansion capabilities of current devices, sometimes several devices are needed to fully block the lumen.

Cardiovascular stents are synthetic material scaffolds used to expand and/or support blood-carrying vessels. The first clinical application of a metallic stent was performed in 1986. Since this pioneering surgery, approximately 650,000-1,000,000 percutaneous coronary interventions (PCI) are performed each year with nearly 80% of procedures involving stents. In many operations, stents are the standard of care since they can be delivered via minimally invasive surgery resulting in rapid recovery time and less surgical risk.

Stenosis is the constriction or narrowing of an artery often caused by arteriosclerosis, in which cholesterol plaque builds on the inner walls of the artery. Angioplasty is used expand the walls of a stenosed artery using the inflation of a small balloon. However, restenosis occurs in 30-60% of all patients who undergo balloon angioplasty alone within the first 6 months of the procedure. Stents are used in part to reduce the rate of restenosis, which is the re-narrowing of a vessel after widening the vessel. Restenosis after balloon angioplasty follows a 3-stage response: acute elastic recoil, negative remodeling, and neointimal proliferation. Stenting mitigates the responses of acute elastic recoil and negative remodeling and thus reduces the restenosis rate sometimes to as low as 10-40%. However, even in the presence of a stent, neointimal proliferation remains a contributing factor of restenosis and can be caused by the stretching and damaging of the wall during angioplasty, the body's response to the stent material, and a compliance mismatch between the stent and artery.

Metal stents have limited flexibility compared to the wall of some body lumens (e.g., arterial wall), and thus induce a significant compliance mismatch. Even when the overall structural compliance of the metallic stent is matched to the compliance of the body lumen, local stiffness mismatch can cause tiny stent ribs to exert significant local pressure on the body lumen wall.

Shape memory polymer (SMP) materials offer the ability to activate with a mechanical force under the application of a stimulus to adapt to physiological conditions. The stimulus may be light, heat, other types of energy, or other types of stimuli known in the art. Therefore, stents formed of SMPs have the ability to activate with a mechanical force under the application of a stimulus, such as light, heat or other types of stimuli.

### SUMMARY

Medical devices constructed of shape memory polymer (SMP) materials are disclosed herein. These SMP devices are capable of assuming a memory shape at physiological temperatures and may be used in surgical procedures, in both cardiovascular and non-vascular applications. These SMP devices have a post-implantation memory shape that is substantially identical to the insertion site, or have a unique functional shape, and may adapt to the vessel growth or size changes as needed. In one embodiment, a medical device is a SMP stent. In another embodiment the medical device is an SMP vessel occlusion device, such as a plug.

### A BRIEF DESCRIPTION OF THE DRAWINGS

FIG. 1 illustrates chemical structures of typical monomers used in SMP formulation including poly(ethylene glycol) dimethacrylate (PEGDMA), diethyleneglycol dimethacrylate (DEGDMA), and tert-butyl acrylate (tBA); a photoinitiator 2,2-dimethoxy-2-phenyl-acetophenone; and a hydrogel 2-hydroxyethyl methacrylate (2-HEMA);

FIG. 2A illustrates linear possibilities of glass transition temperatures (T_{g}) for three different crosslinkers;

FIG. 2B illustrates a range of possibilities of glass transition temperatures by mixing different crosslinking monomers;

FIG. 3 illustrates the use of three homopolymers, which allow a greater range of modulus to T_{g} relationships;

FIG. 4 illustrates the recovery times of a 22 mm stent at body temperature wherein T_{g} and weight percent (wt. %) crosslinking were varied;

FIG. 5 illustrates a typical glass transition curve with T_{g} marked by the peak of tan delta;

FIG. 6 illustrates two glass transition curves with 10 wt % and 20 wt % crosslinking;

FIG. 7 illustrates the recovery forces of a 10 wt % crosslinked sample (average molecular weights Mn=875) and a 20 wt % crosslinked sample (Mn=550) wherein both samples were compressed 30% and had T_{g}=50° C.;

FIG. 8 illustrates pressure and diameter curves of a polymer tube obtained by ultrasonic imaging as a function of time;

FIG. 9 illustrates the glass transition and rubbery modulus as a function of increasing wt % of PEGDMA (550) crosslinker;

FIG. 10 illustrates the free recovery results of four stents with different T_{g}'s and wt % crosslinking;

FIG. 11 illustrates the free recovery results of a solid and a porous stent with similar T_{g} and wt % crosslinking;

FIG. 12 illustrates free recovery data of solid and porous stents made from 3 different compositions and compacted at room temperature;

FIG. 13 illustrates the effect of free recovery activation with respect to T_{g} and packaging temperatures (T_{d});

FIG. 14 illustrates X-ray images of 3 stents having 4% iodopamide around the walls of the stents with a thin gold foil membrane inserted into a stent wall;

FIG. 15 is a cross-sectional, B-mode, ultrasound image of a stent within a water bath taken using 7.5 MHz imaging frequency;

FIG. 16 illustrates sequential images from a stent crush-recovery experiment;

FIG. 17 is a flow chart of an embodiment of a method of controlling SMP properties via variations in a crosslinker in the SMP formulation;

FIG. 18 is a flow chart of an embodiment of a method of controlling a property of a SMP via variations in a molecular weight and wt. % crosslinker in the SMP formulation;

FIG. 19 illustrates an exemplary computer program that implements some of the methods described herein;

FIG. 20 is a graph of relationships between glass transition temperature and percentage weight crosslinker for various Mn of crosslinker;

FIG. 21 is a graph of relationships between glass transition temperature and molecular weight of crosslinker for various wt. % of crosslinker;

FIG. 22 is a graph of relationships between rubbery modulus and wt. % of crosslinker;

FIG. 23 is a graph of rubbery modulus versus molecular weight of crosslinker for various wt. % of crosslinker;

FIG. 24 is a graph of exemplary relationships between modulus and temperature illustrating the modulus transition of three different exemplary SMP networks as manufactured;

FIG. 25 is another graph of exemplary relationships between modulus and temperature illustrating the modulus transition of four different exemplary SMP networks;

FIG. 26 is a graph of recovery percentage versus time for various wt. % of crosslinker;

FIG. 27 is a graph of modulus versus temperature illustrating the modulus transition of an exemplary SMP network;

FIG. 28 is a graph of recovery percentage versus time for three different SMP networks, each with a different wt. % crosslinker and/or a different T_{g};

FIG. 29 is the distinction between recovery time characteristic and actual recovery time, by showing a number of SMP networks, each with different T_{g} responding to similar recovery stimuli;

FIG. 30 is a flow chart of a method of manufacturing SMP devices;

FIG. 31 is a flow chart of an embodiment of a method of determining a recovery time;

FIG. 32 is a flow chart of an embodiment of a method of determining a manufacturing parameter based on a patient characteristic;

FIG. 33 illustrates a smooth surface comprising a SMP network and heparin particles;

FIG. 34 illustrates a significant increase in surface variation after heparin has been removed both from the combined surface of a SMP network and heparin, and from the body of the SMP network;

FIG. 35A is a graph of normalized strain versus time for a recovery temperature Tᵣ=T_{g};

FIG. 35B is a graph of normalized strain versus time for Tᵣ=0.875*T_{g};

FIG. 35C is a graph of normalized strain versus time for a recovery temperature, Tᵣ=0.75*T_{g};

FIG. 36 is a flow chart of an embodiment of a method for achieving a peak stress in a SMP during the recovery phase of the SMP via variations in the deformation temperature of the SMP during manufacturing;

FIG. 37 illustrates an exemplary solid SMP stent;

FIGS. 38A-38C illustrate various exemplary fenestrated SMP stents having between 10%-50% of wall material removed;

FIGS. 39A-39C illustrate sequential images of recovery expansion of a slit SMP stent;

FIGS. 40A-40F illustrate sequential images of deployment (by a catheter) and expansion of a solid SMP stent having circumferential radiopaque ribs;

FIGS. 41A-41D illustrate sequential images of coiling recovery expansion of a coiled SMP stent;

FIGS. 42A-42D illustrate sequential images of deployment (by a catheter) and expansion of a coiled SMP within a clear tube;

FIGS. 43A-43E illustrate sequential cross-sectional images of recovery expansion of a solid or fenestrated SMP stent;

FIG. 44 illustrates a fully-expanded, bulb-shaped SMP plug forming a liquid-tight seal within the lumen of a vessel;

FIGS. 45A-45C illustrate sequential images of deployment (by a catheter) and expansion of a bulb-shaped SMP plug within the lumen of a vessel;

FIG. 46 illustrates a fully expanded bulb-shaped SMP plug having an elongated tail portion;

FIGS. 47A-47E illustrate various additional shapes and designs for SMP plugs;

FIGS. 48A and 48B illustrate SMP plugs having a wire or wires formed therein;

FIGS. 49A-49D illustrate SMP plugs having hooks, anchors or barbs;

FIG. 50 illustrates various different shapes for SMP plugs;

FIG. 51 illustrates various different cross-sectional designs for vessel occlusion plugs or partial vessel occlusion plugs;

FIG. 52 illustrates a cross-sectional view of the unrolling and expansion of a coiled SMP plug;

FIG. 53 illustrates a cross-sectional view of the swelling of a coiled SMP plug having a hydrogel material therein; and

FIG. 54 illustrates an exemplary coiled SMP plug having swollen hydrogel material fully occluding a clear tube.

### DETAILED DESCRIPTION

Medical devices constructed of shape memory polymer (SMP) materials are disclosed herein. In one embodiment, the medical devices may comprise stents having a number of different configurations. The stents disclosed herein may be used for vascular stenting applications, such as for cardiovascular purposes to support or increase the diameter of the lumen of a vessel affected by narrowing. The stents may also be used for non-vascular applications wherein a stent is used to maintain or increase the diameter of the lumen of a vessel affected by narrowing. In other embodiments, the medical devices may comprise plugs having a number of different configurations. The plugs disclosed herein may be used for vascular purposes, such as for cardiovascular purposes to occlude or block the lumen of a vessel to prevent fluid passage through the vessel. The plugs may also be used for non-vascular purposes, such as to block or occlude the lumen of a non-blood-carrying vessel.

Both the stents and plugs disclosed herein may be formed of the SMP materials, which are capable of assuming a memory shape at physiological temperatures and may be used for in vivo medical applications. The SMP stents and plugs disclosed herein have a post-implantation memory shape that is substantially identical to the insertion site, or is shaped in another functional form. These SMP stents and plugs may be designed with specific shape memory and thermomechanical properties that can be tailored to or around different physiological conditions.

The SMP stents and plugs disclosed herein may be formed from a first monomer and a second crosslinking monomer. The weight percentages of the first monomer and second monomer may be selected by performing an iterative function to reach predetermined thermomechanical properties, such as glass transition temperature (T_{g}) and rubbery modulus, for example. Other thermomechanical properties consider in determining the weight percentages of the first and second monomer may include a desired predeformation temperature (T_{d}), storage temperature (Tₛ), recovery temperature (Tᵣ), or deployment time. The selection of the weight percentages of the first and second monomers may optimize the post-implantation memory shape properties of the SMP stents and plugs. References to a crosslinker, crosslinking, or crosslinking density herein refer to the final weight percentage of a crosslinking monomer in the final polymer formulation (before polymerization).

The technology disclosed herein utilizes SMP materials, as disclosed in U.S. Provisional Application Ser. No. 60/788,540 entitled Shape Memory Polymer Medical Device and in International PCT Application No. PCT/US2006/060297 entitled A Polymer Formulation A Method of Determining A Polymer Formulation and A Method of Determining a Polymer Fabrication, which are both hereby incorporated herein by reference for all that they disclose.

Some general properties and advantages of SMP stent and plug devices will first be described below, followed by exemplary polymers used to fabricate SMPs and the methods of determining polymer formulations for use in SMP stents and plugs, followed by discussion of the various SMP stent and plug device embodiments and methods of manufacturing these SMP stents and plugs (including examples).

### General Properties and Advantages of SMP Stents and Plugs

Several types of SMP cardiovascular stent and plug occluding devices are disclosed herein. These SMP stents and plugs may be formed of polymers having shape memory and thermomechanical properties that can be tailored to or around physiological conditions.

The thermomechanical behavior of the stents and plugs can be optimized to physiological conditions by controlling the modulus, visco-elastic properties, and damping coefficient (tan delta). These properties can be tailored to allow the stent and plug diameter to increase as vessel diameter increases, thereby allowing the stent and/or plug to grow with the patient, a property that is particularly useful for pediatric applications. In some embodiments, the entire stent or plug may be composed of a polymer allowing for significantly larger quantities of anti-thrombogenic, anti-inflammatory, or other drugs to be packaged within the stent or plug for release over longer periods of time.

SMPs have the ability to recover large strains after significant mechanical deformation upon an increase in temperature. This shape memory effect allows the stents and plugs to be packaged to a size much smaller than the original state and delivered via much smaller catheters than those currently used. The use of the small catheters and the small delivery size of the SMP stents and plugs provides a less-invasive delivery method for deploying these SMP stents and plugs within a patient. Once inserted into a patient, the stents and/or plugs may then deploy or expand back to the original state with a change in stimuli, such as an increase in temperature supplied either by the body or an external device. The temperature and rate at which the stents and plugs are deployed may be controlled by the chemistry and structure of the polymers, as will be described in more detail below.

Cardiovascular interventionalists, surgeons, and radiologists serving both the adult and pediatric populations may find these SMP stents and plugs to be advantageous because: 1) these devices can be inserted using smaller catheters and still expand into larger blood vessels than current devices; 2) the mechanical properties of these devices can be pre-configured based on the requirements of the treatment; 3) the loading capability of the polymer stents and plugs for anti-thrombotic, anti-inflammatory, or other drugs will be higher than polymer-coated metal stents; and 4) pediatric cardiologists will appreciate the capability of the stent or plug to grow with blood vessel or septal size. These properties of the disclosed SMP stents and plugs provide corresponding advantages over currently employed metal stents and plugs.

In addition, urologists, pulmonologists, and interventionalists working with the biliary, esophageal, urinary, gynecological, pulmonary, hepatic and other non-vascularsystems would find the disclosed SMP stents and plugs useful for many of the same reasons mentioned above. Also, in some of these systems, the target vessel may be large, requiring a large stent, but the treatment catheter should remain as small as possible. The disclosed SMP stents and plugs maximize this capability by allowing extremely large (>200%) changes in stent or plug diameter upon deployment or expansion. (Note that the attached drawings illustrating recovery or expansion are for purposes of exemplary illustration only and some of the figures shown herein may not be to scale.)

The SMP stents and plugs disclosed herein may be primarily polymer based, which may provide significantly greater volume of polymer for attaching or suspending various drugs. For example, the polymer stents and plugs may have small pockets containing anti-thrombolytic, anti-proliferative, or other desirable agents. These pockets may be broken to release the drug using balloon dilation after deployment, or they may be designed to rupture during shape recovery upon initial deployment. As another example, certain drugs may be immobilized on the surface or within the polymer; these drugs would then diffuse out of the polymer slowly over time. The disclosed stents and plugs thus have the ability to infuse greater amounts of drugs into the polymer than with current polymer-coated metal stents.

Basic thermomechanical response of shape memory polymers is defined by four critical temperatures. The glass transition temperature, T_{g}, is typically represented by a transition in modulus-temperature space and can be used as a reference point to normalize temperature. Shape memory polymers offer the ability to vary T_{g} over a temperature range of several hundred degrees by control of chemistry or structure. The predeformation temperature, T_{d}, is the temperature at which the polymer is deformed into its temporary shape. The storage temperature, Tₛ, represents the temperature in which no shape recovery occurs and is equal to or below T_{d}. At the recovery temperature, Tᵣ, the shape memory effect is activated, which causes the material to recover its original shape, and is typically in the vicinity of T_{g}. Recovery can be accomplished isothermally by heating to a fixed Tᵣ and then holding, or by continued heating up to and past Tᵣ.

The microscopic mechanism responsible for shape memory in polymers depends on both chemistry and structure of the polymers. If the polymer is deformed into its temporary shape at a temperature below T_{g}, or at a temperature where some of the hard polymer regions are below T_{g}, then internal energy restoring forces will also contribute to shape recovery. In either case, to achieve shape memory properties, the polymer must have some degree of chemical crosslinking to form a “memorable” network or must contain a finite fraction of hard regions serving as physical crosslinks.

More than one method may be used to design SMP for use in the stent and plug medical devices disclosed herein. In one exemplary method, the polymer transition temperature may be tailored to allow recovery at the body temperature, Tᵣ˜T_{g}˜37° C. (A. Lendlein and R. Langer, “Biodegradable, elastic shape-memory polymers for potential biomedical applications.” Science, vol. 296, pp. 1673-1676, 2002). The distinct advantage of this approach is the utilization of the body's thermal energy to naturally activate the material. The disadvantage of this approach, for some applications, is that the mechanical properties (e.g., stiffness) of the material are strongly dependent on T_{g}, and would be difficult to alter in the device design process. In particular, it would be difficult to design an extremely stiff device when the polymer T_{g} is close to the body temperature due to the compliant nature of the polymer. Another possible disadvantage is that the required storage temperature, Tₛ, of a shape memory polymer with T_{g}˜37° C. will typically be below room temperature requiring “cold” storage prior to deployment.

In an alternative exemplary method, the recovery temperature may be higher than the body temperature Tᵣ˜T_{g}>37° C. (M. F. Metzger, T. S. Wilson, D. Schumann, D. L. Matthews, and D. J. Maitland, “Mechanical properties of mechanical actuator for treating ischemic stroke,” Biomed. Microdevices, vol. 4, no. 2, pp. 89-96, 2002; D. J. Maitland, M. F. Metzger, D. Schumann, A. Lee, T. S. Wilson, “Photothermal properties of shape memory polymer micro-actuators for treating stroke.” Las. Surg. Med., vol. 30, no. 1, pp. 1-11, 2002). The advantage of this second method is that the storage temperature can be equal to room temperature facilitating easy storage of the device and avoiding unwanted deployments prior to use. The main disadvantage of this second method, for some applications, is the need to locally heat the polymer to induce recovery. Local damage to some cells in the human body commences at temperatures approximately 5 degrees above the body temperature through a variety of mechanisms including apoptosis and protein denaturing. Advocates of the second approach use local heating bursts to minimize exposure to elevated temperatures and circumvent cell damage. The use of one method over the other is a design decision that depends on the targeted body system and other device design constraints such as required in-vivo mechanical properties.

A polymer is a shape memory polymer (SMP) if the original shape of the polymer can be recovered by application of a stimulus, e.g., by heating it above a shape recovery temperature, or deformation temperature (T_{d}), even if the original molded shape of the polymer is destroyed mechanically at a lower temperature than T_{d}. The original shape is set by processing and the temporary shape is set by thermo-mechanical deformation. A SMP has the ability to recover from large deformation upon heating. The present devices are made from SMPs, which can subsequently be deformed or crushed and inserted into a vessel lumen, or other aperature or cavity, and then be deployed or expanded by an increase in temperature, for example, to hold a new fixation graft, occlude a vessel, or plug a septal defect. The ability for these devices to be deformed provides a benefit of easy installation through optimal compacted loading configurations. In certain embodiments, the shape may be smooth in texture. In other embodiments, the shape may range from smooth to fully textured. In alternative embodiments, the shape may be partially textured.

SMPs have selective biocompatibility with different areas of the body. For example, FDA approved dental materials may not be biocompatible in a cardiovascular environment. As another example, polyethyleneglycol (PEG), also known as polyethylene oxide (PEO), has been studied for its protein and cell resistance, which renders a non-fouling surface. Therefore, the polymers used to construct the SMP stents and plugs for cardiovascular applications need to be carefully tailored to each specific application. SMP applications of biocompatible SMPs, which capitalize on observed thermomechanical behaviors, include medical devices such as cardiovascular stents, septal defect plugs, non-cardiovascular stents, and vessel occlusion plugs or systems.

### Methods of Determining Polymer Formulations for Use in SMP Stents and Plugs

The properties of the SMP stents and plugs disclosed herein may be controlled by changing the formulation of the polymers, or by changing the treatment of the polymers through polymerization and/or handling after polymerization. Disclosed herein are methods for determining polymer formulations to achieve these different properties or characteristics.

The techniques of controlling SMP properties rely on an understanding of how SMP properties are affected by these changes and how some of these changes may affect more than one property. For example, changing the percentage weight of a crosslinker in a SMP formulation may change both a transition temperature of the SMP and a modulus of the SMP, as mentioned above. In one embodiment, changing the percentage weight of a crosslinker will affect the glass transition temperature and the rubbery modulus of an SMP. In another embodiment, changing the percentage weight of crosslinker will affect a recovery time characteristic of the SMP.

Some properties of a SMP may be interrelated such that controlling one property has a strong or determinative effect on another property, given certain assumed parameters. For example, the force exerted by a SMP against a constraint (e.g., a bony tunnel or a body lumen) after the SMP has been activated may be changed through control of the rubbery modulus of the SMP. Several factors, including a level of residual strain in the SMP enforced by the constraint will dictate the stress applied by the SMP, based on the modulus of the SMP. The stress applied by the SMP is related to the force exerted on the constraint by known relationships.

Examples of constituent parts of the SMP formulation include monomers, multi-functional monomers, crosslinkers, initiators (e.g., photo-initiators), and dissolving materials (e.g., drugs, salts). Two commonly included constituent parts are a linear chain and a crosslinker, each of which are common organic compounds such as monomers, multi-functional monomers, and polymers.

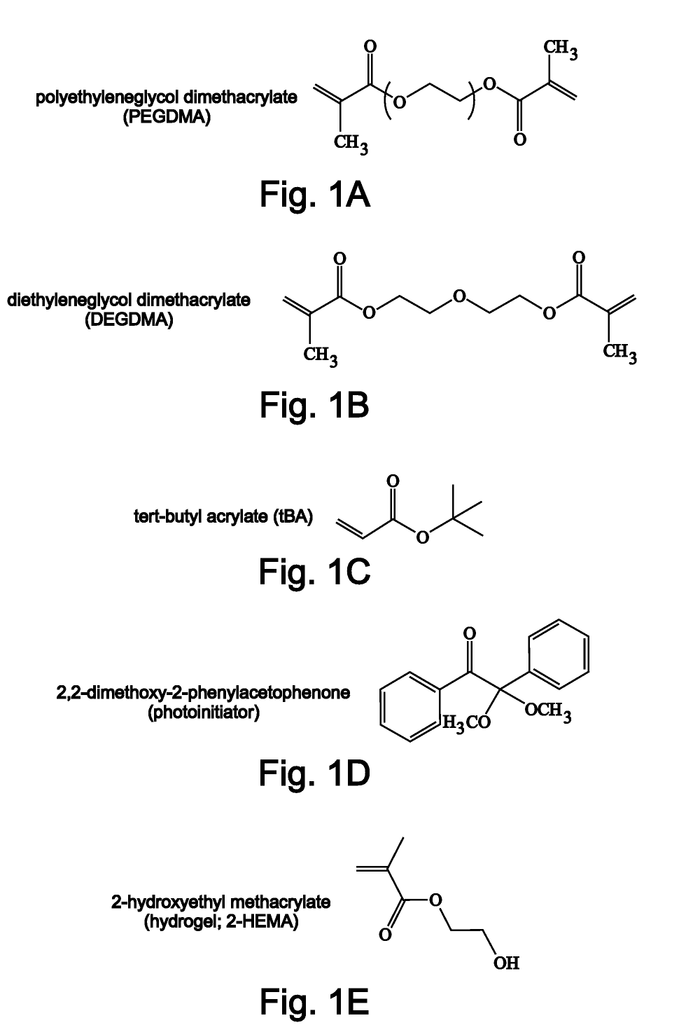

A crosslinker, as used herein, may mean any compound comprising two or more functional groups (e.g., acrylate, methacrylate), such as any poly-functional monomer. For example, a multi-functional monomer is a poly ethylene glycol (PEG) molecule comprising at least two functional groups, such as di-methacrylate (DMA), or the combined molecule of PEGDMA, as shown in FIG. 1. The percentage weight of crosslinker indicates the amount of the poly-functional monomers placed in the mixture prior to polymerization (e.g., as a function of weight), and not necessarily any direct physical indication of the as-polymerized “crosslink density.”

A linear chain may be selected based on a requirement of a particular application because of the ranges of rubbery moduli and recovery forces achieved by various compositions. In one embodiment, a high recovery force and rubbery modulus may be used in an orthopedic graft fixation device comprising a shape memory polymer made from a formulation with methyl-methacrylate (MMA) as the linear chain. In another embodiment, a lower recovery force and rubbery modulus may be used for a body lumen endoprosthesis (e.g., stents and/or plugs) comprising a SMP made from a formulation with tert-butyl acrylate (tBA) (shown in FIG. 1) as the linear chain. In other embodiments, other linear chains may be selected based on desired properties such as recovery force and rubbery modulus.

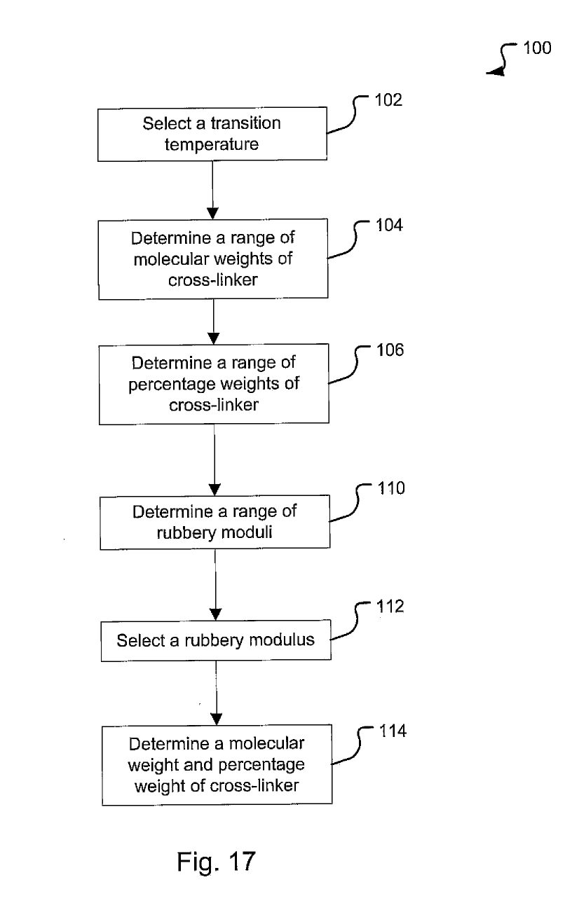

FIG. 17 shows a flow chart of an embodiment of a method 100 of controlling SMP properties via variations in a crosslinker in an SMP formulation. The embodiment shown of method 100 includes selecting a transition temperature 102. The embodiment of the method 100 includes determining a range of average molecular weights 104 of crosslinker material for use in an SMP. A range is determined from the transition temperature selected in 102. The transition temperature may be a desired transition temperature for use in a human body. Such a transition temperature may be close to human body temperature. The transition temperature affects the range of possible average molecular weights of crosslinker material that may be used in the SMP because certain combinations of average molecular weights and of percentage weights of crosslinker produce certain transition temperatures and other combinations produce other transition temperatures.

The method 100 also includes determining a range of percentage weights 106 of crosslinker material for use in an SMP. This range is determined from the transition temperature selected in 102 in a similar manner to that described above for determining a range of average molecular weights. Certain combinations of average molecular weights of crosslinker and percentage weights of crosslinker may be used in the SMP formulation to achieve a certain transition temperature, as described above. These values of percentage weights constitute the range determined in 106.

Determining the range of percentage weight crosslinker 106 and the range of molecular weights 104 is performed based a relationship between transition temperature, molecular weight, and percentage weight crosslinker. The relationship is specific to the linear chain and crosslinker used. Other inputs or manufacturing techniques may also affect the relationship and eventual transition temperature of a SMP.

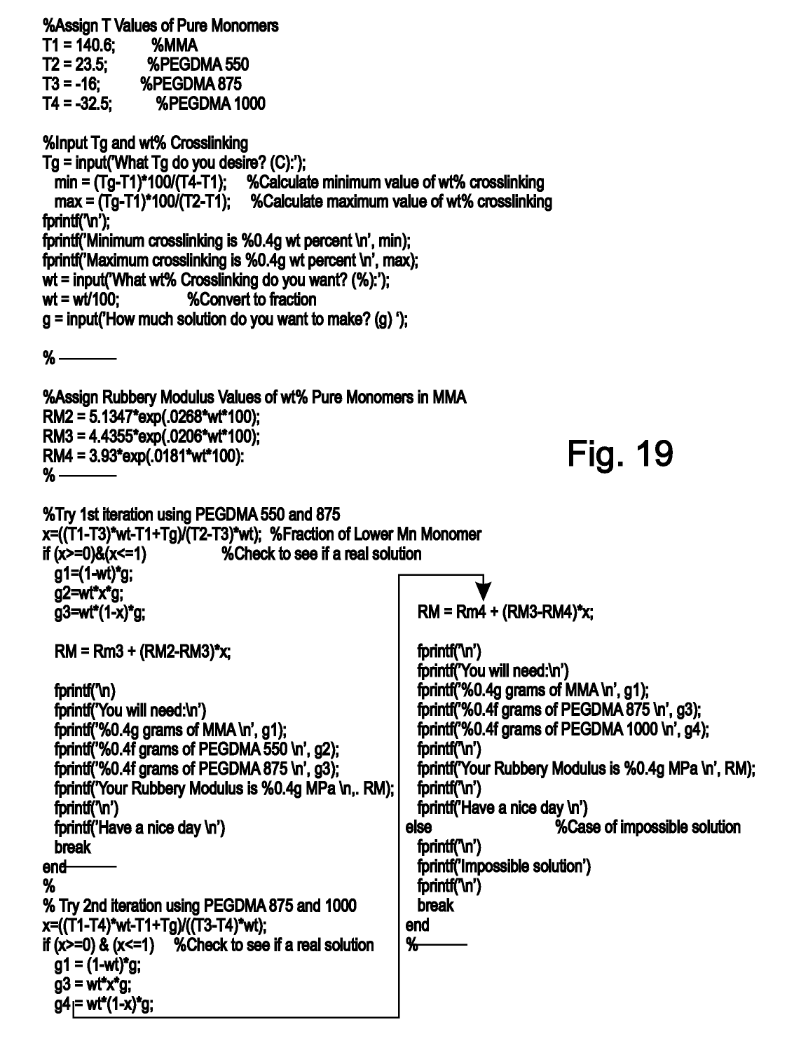

In one embodiment, the method 100 uses empirically-derived relationships which relate molecular weight and weight percentage crosslinker to (a) the transition temperature, (b) the rubbery modulus, and/or (c) a recovery time characteristic. In another embodiment, the method 100 uses relationships which are derived from theoretical models. Examples of empirically-derived relationships are disclosed below and are included within the exemplary computer code in FIG. 19.

The method 100 includes determining a range of rubbery moduli 110 from the ranges of percentage weights and molecular weights. The range of rubbery moduli are determined 110 by evaluating a relationship between the rubbery modulus, percentage weight of crosslinker, and molecular weights for each of the combinations determined in operations 104 and 106. This results in a range of possible rubbery moduli for SMPs which would also have the transition temperature desired and used for operations 104 and 106.

A rubbery modulus is selected 112 from the range of rubbery moduli. In one embodiment, selecting 112 may be performed in response to the range of moduli being determined 110 or before the range is determined, for example, as an initial goal value of rubbery modulus for the SMP. In another embodiment, the selecting 112 may be performed after another transition temperature is selected, producing another range of rubbery moduli. In other words, the method 100 may be performed, for example, iteratively, repeatedly, and/or in parts.

The method 100 also includes determining a molecular weight and percentage weight of crosslinker 114 based on the selected rubbery modulus. In one embodiment, the determining 114 is performed using the relationship between rubbery modulus, molecular weight and percentage weight of crosslinker to find the combination of molecular weight and percentage weight that corresponds to the rubbery modulus selected.

The operations of determining a range of molecular weights and percentage weights of crosslinker (104, 106) may be performed at about the same time. In one embodiment, determining a range of molecular weights 104 may create relationships that may be used to determine a range of percentage weights of crosslinker 106. These two determinations (104, 106) may be performed with any time separation, at about the same time, at large time intervals, or simultaneously.

In another embodiment, determining a range of molecular weights and percentage weights of crosslinker (104, 106) may be performed by creating and/or selecting a table, graph, or chart corresponding to a desired transition temperature or a desired rubbery modulus among a plurality of tables, graphs, and/or charts. In this embodiment, the tables, graphs, and/or charts include information from the relationships described above and outline ranges of molecular weights and percentage weights crosslinker that correspond to the desired value of the property (e.g., transition temperature).

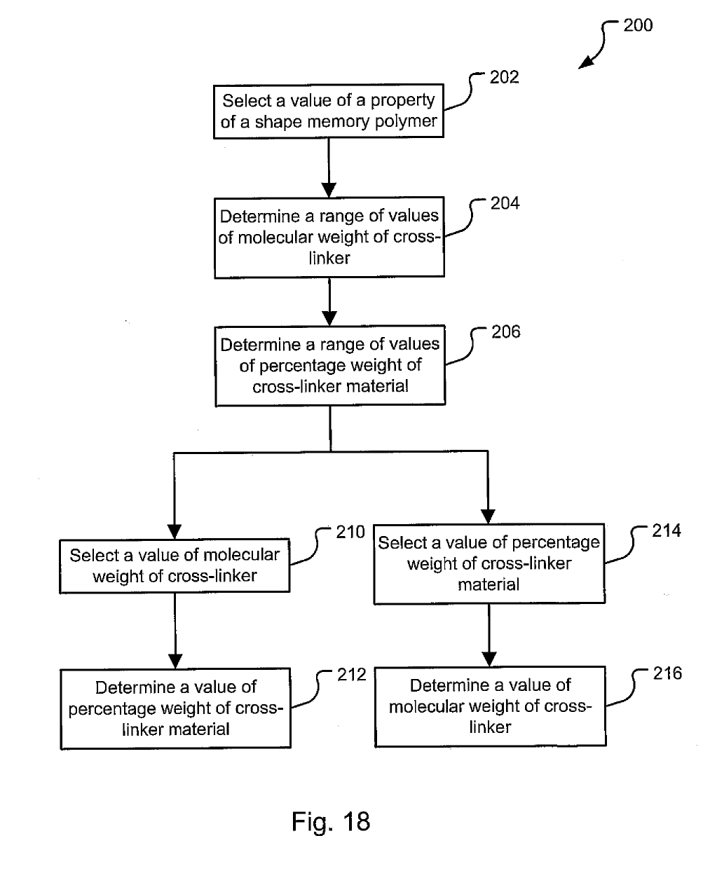

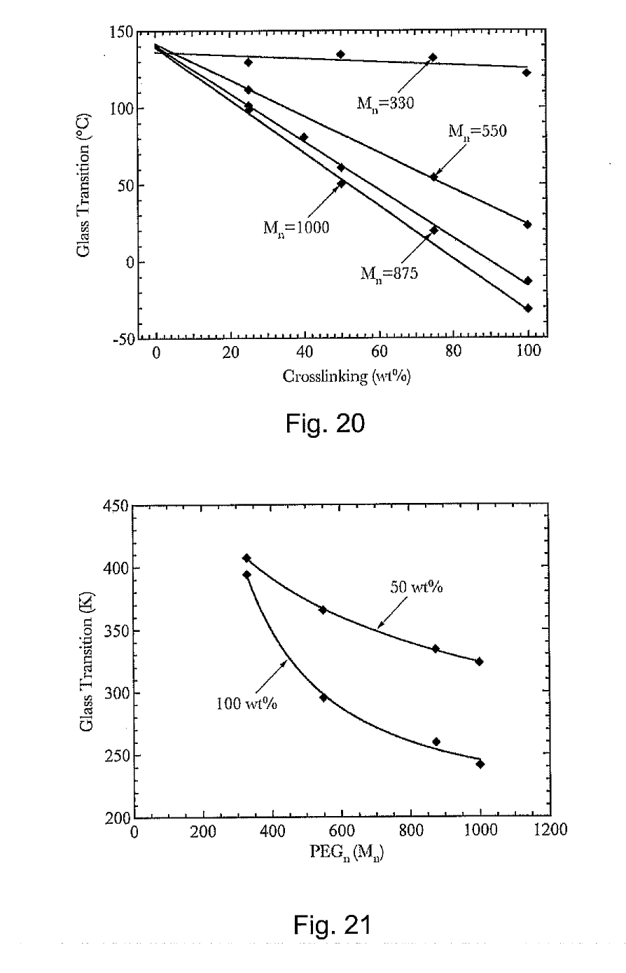

FIG. 18 shows a flow chart of an embodiment of a method 200 of controlling a property of a SMP via variations in a molecular weight and percentage weight crosslinker in the SMP formulation. Method 200 includes selecting a value 202 of a property of a SMP. In this embodiment, only one value of a property (e.g., a desired value of the property for the SMP) is selected. A range of values of molecular weight of a crosslinker is determined 204. Also a range of values of percentage weight of crosslinker is determined 206. Each of these ranges are determined based on relationships which relate the inputs of molecular weight and percentage weight of crosslinker which would attain the value of the property selected in 202. For example, if a certain rubbery modulus is desired for a SMP, a range of percentage weights of crosslinker may be used, and a range of average molecular weights of crosslinker may be used. These ranges may be further understood in the form illustrated in FIGS. 20-25.

The embodiment of the method 200 also includes the option of selecting a value 210 of an average molecular weight of a crosslinker, and determining a value 212 of percentage weight of the crosslinker. The embodiment of the method 200 also includes the option of selecting a value 214 of a percentage weight of a crosslinker, and determining a value 216 of average molecular weight of the crosslinker. Each of these options utilizes the selected value of the property (e.g., from 202) and the selected value of one of the inputs (e.g., percentage weight from 210, average molecular weight from 214) to determine the value of the other input.

FIG. 19 is an example of a computer program that implements some of the methods described herein. The computer program is written in a language compatible with Matlab (The Mathworks, Inc., Natick, Mass.). The program assumes a linear chain of MMA, a crosslinker species of PEGDMA, and average molecular weights of crosslinkers of 550, 875 and 1000. Each of the relationships used in the program were derived from data contained in FIGS. 20-25 and discussed further below.

The program requests a desired glass transition temperature from a user, and receives it from the user's input. From this desired glass transition temperature, a range of possible values is given for percentage weight of crosslinker material. This range is calculated using the temperature coefficients of the “pure” monomers, which denote the average molecular weights of some commercially available monomers (e.g., PEGDMA with molecular weights of 550, 875, 1000). Therefore, this range is limited by the limited number of monomers used for the calculation and a larger range may be possible if other monomer species and/or weights were used by the program (e.g., PEGDMA with a molecular weight of 330).

The program then requests user input regarding the desired percentage weight of crosslinker material within the range supplied. The program then iteratively tries a first group of two crosslinkers (e.g., with molecular weights 550 and 875). In other words, the program checks if an average molecular weight between 550 and 875 will provide the desired glass transition temperature (e.g., a property of the SMP) and the desired percentage weight of crosslinker (e.g., an input value of the SMP network). If so, the mixing ratio of the two average molecular weights is provided along with the resultant rubbery modulus. If not, the program checks if an average molecular weight between 875 and 1000 will work.

Other relationships may be included and/or substituted as empirically or theoretically derived from experimentation with different linear chains and crosslinkers. Some of these other relationships are demonstrated in FIGS. 20-25, which detail empirical data derived for some of the linear chain and crosslinker networks described herein. FIGS. 13-18 show molecular weights of crosslinker as PEGn(Mn) which may include several types of crosslinker molecules. In FIGS. 13-18, PEGn(Mn) refers to PEGDMA of multiple varieties and configurations. For example, these crosslinkers may have included PEG molecules with multiple different molecular structures and configurations with di-methacrylate (DMA). In the experiments for FIGS. 20-25, PEGDMA of the average molecular weight indicated in the graph was used and should be understood as PEGDMA of that molecular weight, rather than pure PEG of that molecular weight.

FIG. 20 is a graph of relationships between glass transition temperature and percentage weight crosslinker for various average molecular weights (Mn) of crosslinker. Each line is a fit of data taken from SMP networks using a single average molecular weight crosslinker. The difference in slope between the lines illustrates the difference in glass transition temperature that may be achieved for any given percentage weight crosslinker by varying the average molecular weight of the crosslinker. In addition, the difference in slope between the lines illustrates the ability to vary the percentage molecular weight of crosslinker for any given glass transition temperature, possibly to achieve values of other properties (e.g., recovery time characteristic of the SMP).

FIG. 21 is a graph of relationships between glass transition temperature and molecular weight of crosslinker for various percentage weights of crosslinker. Each line is a fit from data of a SMP networks with the same percentage weight of crosslinker, but including different molecular weights of the crosslinker. This graph shows another view of some of the same data shown in FIG. 20.

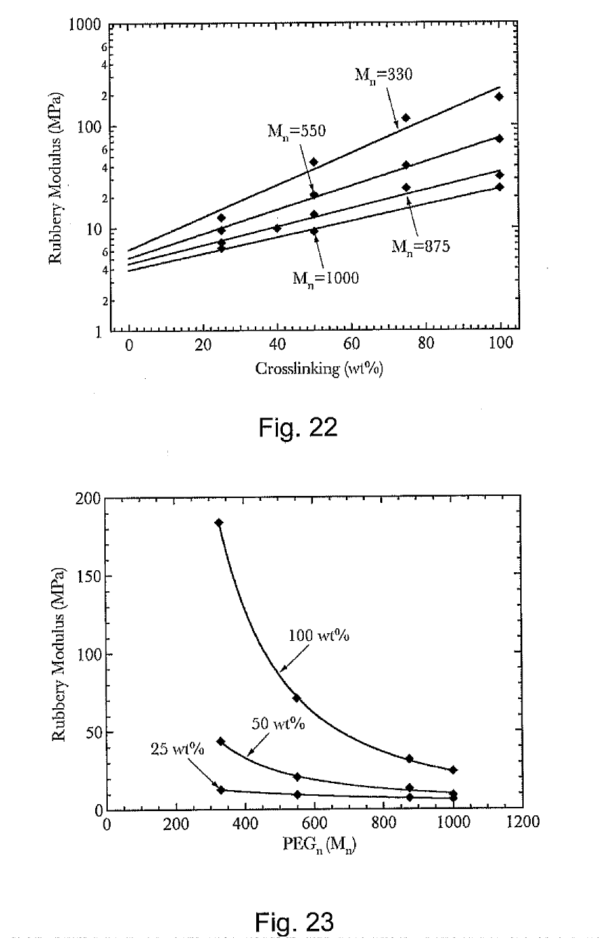

FIG. 22 is a graph of relationships between rubbery modulus and percentage weight of crosslinker. Each curve is a fit of data taken from SMP networks using a single average molecular weight crosslinker. The difference between the curves illustrates the difference in rubbery modulus that may be achieved for any given percentage weight crosslinker by varying the average molecular weight of the crosslinker. A single relationship between rubbery modulus, molecular weight, and percentage weight of crosslinker may be created using the different curves. In addition, the difference between the curves illustrates the ability to vary the percentage weight of crosslinker without varying the rubbery modulus, possibly to achieve values of other properties (e.g., recovery time characteristic of the SMP).

FIG. 23 is a graph of rubbery modulus versus molecular weight of crosslinker for various percentage weights of crosslinker. Each curve is a fit from data of a SMP networks with the same percentage weight of crosslinker, but including different molecular weights of the crosslinker. The curves show relationships between the average molecular weight of crosslinker and the rubbery modulus that may be used to determine other inputs to a formulation given certain desired properties of an SMP network. For example, a particular relationship or curve may be chosen by selecting a percentage weight crosslinker for the SMP network.

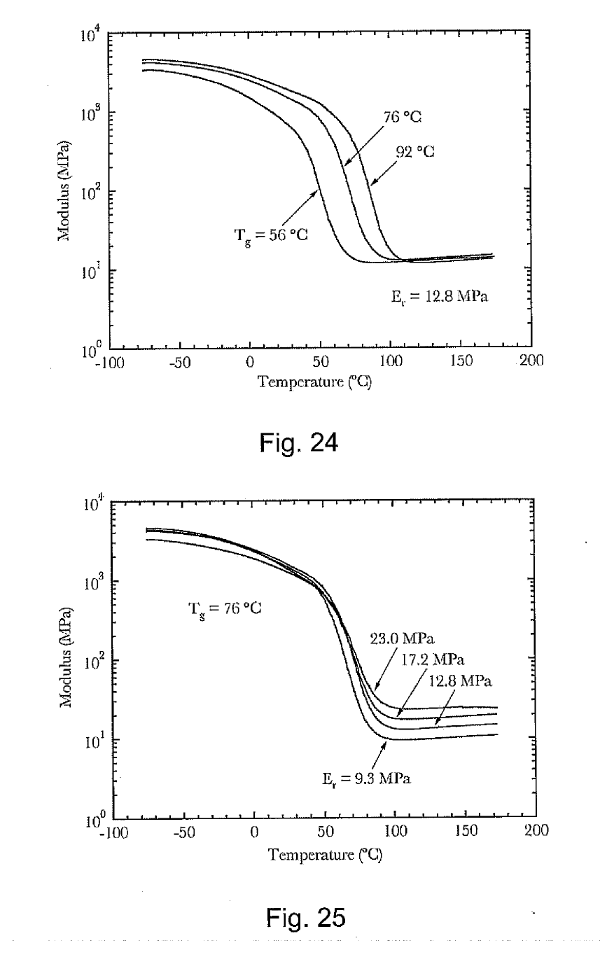

FIG. 24 is a graph of exemplary relationships between modulus and temperature illustrating the modulus transition of three different exemplary SMP networks as manufactured. The graph tracks the change in modulus of the SMP network as the network is cycled from a low temperature (e.g., the storage temperature) to a higher temperature. The change in modulus may indicate, for example, a shape change in the SMP network and/or a stress exerted by the SMP network on an environmental constraint. Any shape change of the SMP network may also be affected by environmental constraints surrounding the network while the network undergoes a modulus transition.

FIG. 24 illustrates the ability, using some of the techniques described herein, to change a glass transition temperature (e.g., from 56 degrees to 92 degrees Celsius) without changing the rubbery modulus of an SMP network (e.g., keeping it fixed at about 12.8 MPa). For example, the SMP network with a glass transition temperature of 56 degrees Celsius has substantially the same rubbery modulus as the SMP networks with glass transition temperatures of 72 degrees and 92 degrees Celsius. Thus, through using some of the techniques herein, glass transition temperature may be varied substantially independently from rubbery modulus.

FIG. 25 is another graph of exemplary relationships between rubbery modulus and temperature illustrating the rubbery modulus transition of four different exemplary SMP networks. FIG. 25 illustrates the ability, using some of the techniques described herein, to change a rubbery modulus (e.g., from 9.3 MPa to 12.8 MPa to 17.2 MPa to 23.0 MPa) without changing the glass transition temperature of a SMP network (e.g., keeping it fixed at about 76 degree Celsius). For example, the SMP network with a rubbery modulus of 12.8 MPa has substantially the same glass transition temperature as the SMP networks with rubbery moduli of 9.3 MPa and 23.0 MPa. Thus, through using some of the techniques herein, rubbery modulus may be varied substantially independently from glass transition temperature.

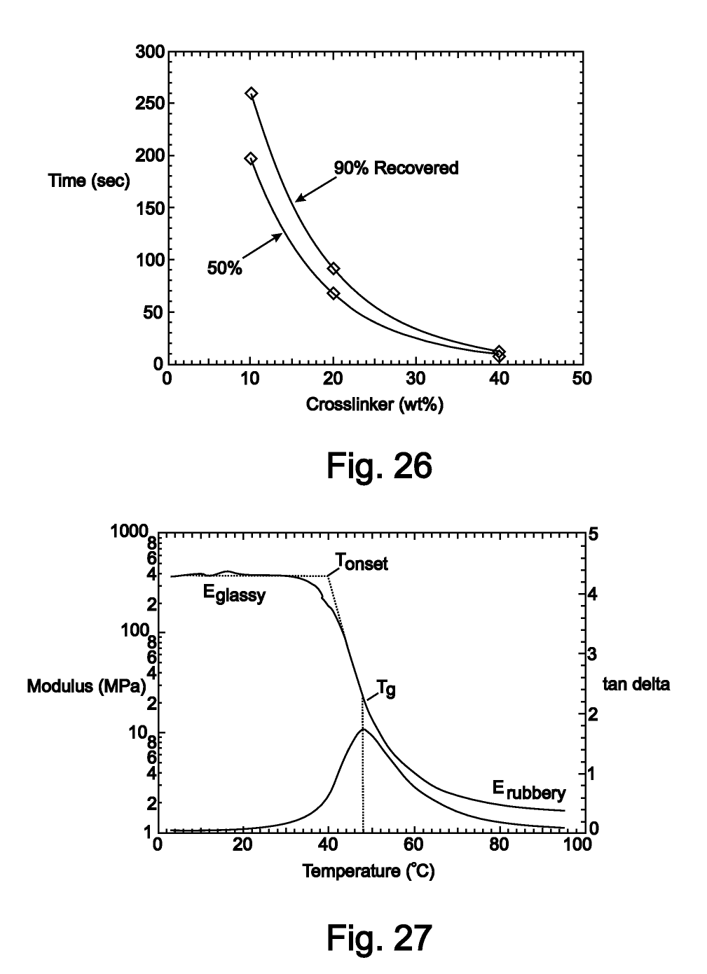

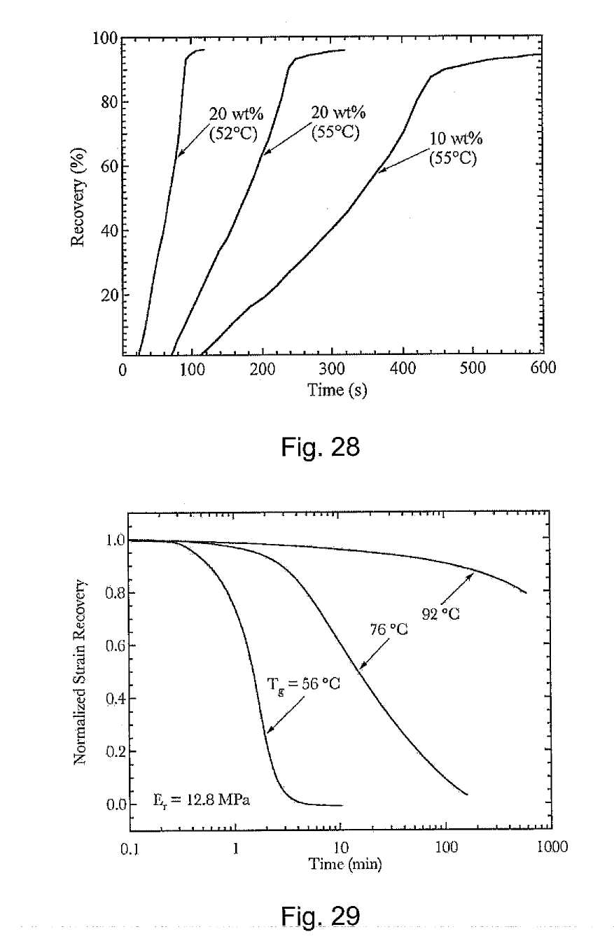

FIG. 26 is a graph of recovery percentage versus time for various percentage weights of crosslinker. Each curve is a fit of data taken from different SMP networks, and represents the time the network took to recover to a certain recovery percentage. Other curves may be derived from FIG. 28 as some of the same data is disclosed in that graph. The difference between the curves illustrates the differences in recovery time characteristics that may be achieved by changing the percentage weight crosslinker. For example, the time difference from 50% recovered to 90% recovered is significantly shorter for 40% weight of crosslinker networks than it is for 10% weight of crosslinker networks. In addition, the overall recovery time to 90% recovered is much shorter between those two networks. These differences in time are achieved despite recovering the networks under similar conditions (e.g., stimulus interface or stimulus magnitude) and are largely the result of the differences in the structure of the network rather than any differences in the recovery environment.

FIG. 27 is a graph of modulus versus temperature illustrating the modulus transition of an exemplary SMP network. The graph also includes a tan-delta measurement of the modulus change and illustrates a method of determining a transition temperature of the material. The method includes finding temperature for the peak tan-delta of the modulus of the material. The tan-delta measurement represents the ratio of the storage modulus (shown), or alternatively, the real part of the modulus of the SMP network under dynamic analysis, to the loss modulus (not shown), or alternatively, the imaginary part of the modulus of the SMP network similarly under dynamic analysis. The graph was produced using a standard three-point flexural test using a dynamic modulus analysis machine (DMA machine).

FIG. 28 is a graph of recovery percentage versus time for three different SMP networks, each with a different percentage weight crosslinker and/or a different glass transition temperature. The three different networks include a network with 20% weight crosslinker and T_{g}=52 degrees Celsius, a network with 20% weight crosslinker and T_{g}=55 degrees Celsius, and a network with 10% weight crosslinker and T_{g}=55 degrees Celsius.

The lines showing different glass transition temperature illustrate the effects of a different T_{g} on actual recovery time. The networks were each recovered using the same magnitude of stimulus (in this instance, temperature of a liquid bath) and the networks also shared the same interface with that stimulus (in this instance, surface area in contact with the bath). The network with a lower T_{g} recovered more quickly than the other networks, in part because the transfer of heat to the lower-T_{g} network (presumably consistent between the networks) caused the lower-T_{g} network to get closer to its Tonset and closer to its T_{g} more quickly than the same transfer of heat did in the higher-T_{g} networks.

FIG. 28 also illustrates that recovery time can be affected by simple energy transfer. Energy transfer is related to the magnitude of a stimulus and the interface the SMP network shares with the stimulus. As introduced above, recovery time characteristics are independent from changes in energy transfer from a stimulus (e.g., heat or light). In other words, changes in T_{g} such as those between the networks in FIG. 28 are not part of a change in recovery time characteristic.

FIG. 28 also illustrates a change in recovery time characteristic between the two SMP networks with T_{g}=55 degrees Celsius. The heat transfer from the stimulus to both of these networks was similar, as was their glass transition temperatures. However, the internal differences between the two networks due to the different percentage weight crosslinker in each of the networks, caused the 10% weight crosslinker network to recover more slowly than the 20% weight crosslinker network.

FIG. 29 illustrates the distinction between recovery time characteristic and actual recovery time, by showing a number of SMP networks, each with different glass transition responding to similar recovery stimuli. The lower T_{g} network (T_{g}=56 degrees Celsius) clearly recovers faster than the higher T_{g} networks. These changes do not necessarily correspond to different recovery time characteristics separate from the different transition temperatures of the three networks. Instead, because the three networks were substantially the same apart from their different transition temperatures, the differences in recovery time between the three networks shows the effect of energy transfer to networks with similar recovery time characteristics, but different transition temperatures.

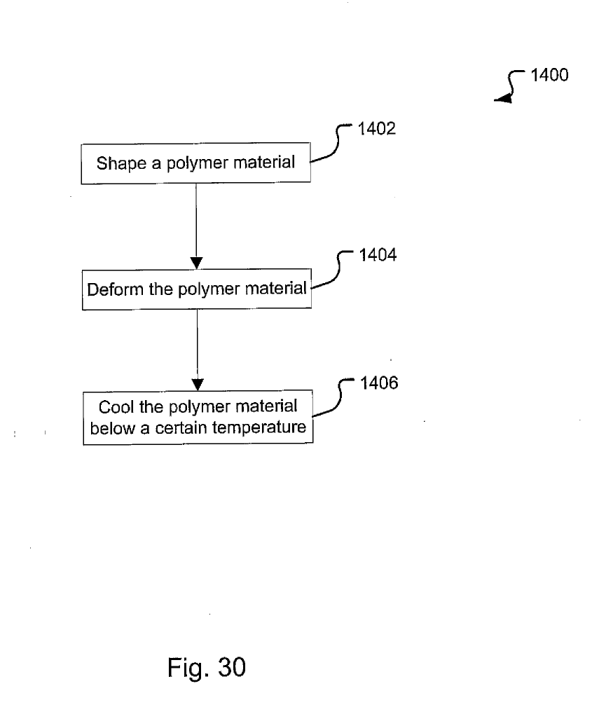

FIG. 30 shows a flow chart of a method 1400 of manufacturing SMP devices. The method 1400 includes shaping a polymer material 1402 into a post-implantation shape, deforming the polymer material 1404 into a pre-implantation shape, and cooling the polymer material 1406 to below a certain temperature.

The method 1400 includes cooling the polymer material 1406 to below a certain temperature. The certain temperature may be the glass transition temperature of the polymer material. In one embodiment, the cooling the polymer material operation 1406 is performed after the deforming the polymer material operation 1404. For example, the polymer material may be above the glass transition temperature while the deforming the polymer material operation 1404 is performed. In another embodiment, the cooling the polymer material operation 1406 is performed before the deforming the polymer material operation 1404.

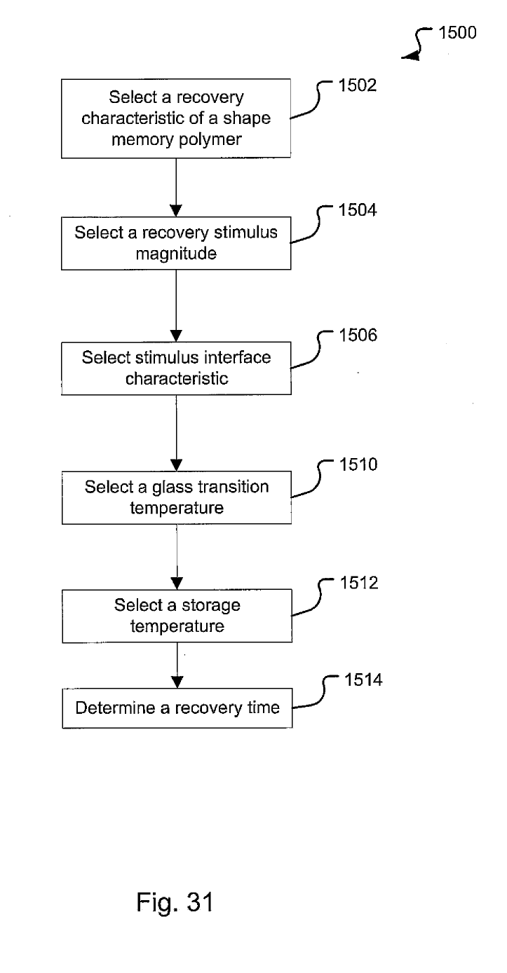

FIG. 31 shows a flow chart of an embodiment of a method 1500 of determining a recovery time. Method 1500 includes selecting a recovery characteristic of a SMP 1502. The method 1500 includes selecting a stimulus magnitude 1504 and selecting a stimulus interface characteristic 1506. The method 1500 also includes selecting a glass transition temperature 1510 and selecting a storage temperature of the SMP 1512. These operations may be used for SMP networks which are heat activated as temperature properties and measurements may or may not be important in SMP applications using a stimulus other than heat (e.g., light or other radiation). A transition temperature and a storage temperature (or other temperature at which the SMP will be held before being subjected to stimulus) may be utilized, through standard thermodynamic calculations, to determine a prediction of the recovery time (1514) of the SMP given the glass transition temperature and storage temperature selected (1510, 1512).

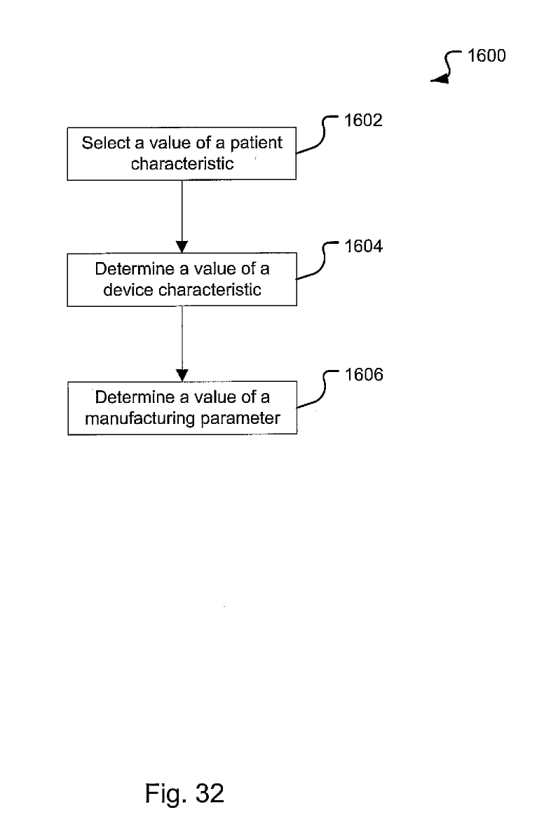

FIG. 32 shows a flow chart of an embodiment of a method 1600 of determining a manufacturing parameter based on a patient characteristic. Using some of the techniques described above, SMP devices may be designed to have properties which are specifically targeted for use with a particular patient. Method 1600 includes selecting a value of a patient characteristic 1602 that relates to a particular patient. Patients receiving therapeutic treatment including a SMP may benefit from specific values of some of the properties of the SMP.

The value of the patient characteristic may be selected (e.g., 1602) from any available source, including via observation of the patient, retrieval from a data store, or reference to a preferred or common value. The value of the patient characteristic may also be any type of patient data measured from a patient, including, for example, data measured by a physician in observing a patient, data recorded by an instrument, data recorded by hand, or observed but not recorded by a physician.

Method 1600 includes determining a value of a device characteristic 1604 based on the selected value of a patient characteristic. In one embodiment, determining a value of a device characteristic 1604 may be performed by matching the value of the device characteristic to the value of the patient characteristic. In another embodiment, determining a value of a device characteristic 1604 may be performed by correlating the value of the patient characteristic with a different value of a device characteristic based on other information (e.g., physician's experience or a correlation table).

Method 1600 also includes determining a value of a manufacturing parameter 1606, which may be performed in any of the manners described above for determining a SMP formulation input based on a desired or selected SMP property. In one embodiment, other inputs of a SMP formulation may be determined using the SMP device characteristic selected (in 1604). In another embodiment, other device characteristics (e.g., other SMP properties) may be used in addition to the device characteristic selected (in 1604) to determine a SMP formulation, depending on, for example, available inputs or other manufacturing factors described above.

A SMP network may include dissolving materials which may include part of the network or may be included in the formulation of the network before the network is polymerized (e.g, as an aggregate or mixed into the formulation). Dissolving materials may include materials that disperse over time, even if the material or part of the material does not actually dissolve or enter into a solution with a solvent. In other words, a dissolving material as used herein may be any material that may be broken down by an anticipated external environment of the polymer. In one embodiment, a dissolving material is a drug which elutes out of a SMP network. A dissolving material may be attached by chemical or physical bonds to the polymer network and may become disassociated with the polymer network over time.

Dissolving materials, through their dissolution over time, may be used for many purposes. In one embodiment, the dissolution of a material may affect a dissolution or breaking up of a biomedical device over time. In another embodiment, the dissolution of a material may elute a drug, achieving a pharmacological purpose. Medications or drugs can be infused into the SMP devices to aid in prevention of clotting. In some embodiments medications or drugs may be coated onto surfaces of the SMP devices.

In some embodiments the matrix of the SMP-based material may be supplemented with a variety of drugs during the polymerization process or post-processing. For example, drugs to be added may include anti-inflammatory, pro-contraceptive, and anti-thrombotic drugs. These drugs can be added by injection into the liquid polymer before UV curing. Drugs may also be added to the SMP devices post-polymerization using various surface modification techniques such as plasma deposition, for example. SMP device design may allow greater amounts of drugs to be infused into the polymer than with current polymer-coated metal stents or plugs.

Dissolving materials may be used to create surface roughness, for example, in order to increase biocompatibility of the network. In one embodiment, the dissolving material may initially form a part of the surface of the SMP network and leave behind a rougher SMP surface after the dissolving material has dissolved. In another embodiment, the dissolving material may be placed within the body of the SMP network and upon dissolving may create an impression in the surface of the SMP by allowing the SMP to collapse due to the dissolution of the dissolving material within the body of the SMP.

An initial surface of an exemplary SMP device may be a rough surface. In one embodiment, an initial rough surface may include a dissolving material. In another embodiment, an initial rough surface may be created by including dissolving material inside a SMP network. Once the material has dissolved, a surface with a different roughness may be left behind. In one embodiment, a smooth surface is left after a dissolving material has dissolved. In another embodiment, a surface rougher than the initial is left behind after a dissolving material has dissolved. In another embodiment, a surface with a different type of roughness is left after a dissolving material has dissolved. For example, an initial surface may have roughness in a random pattern and a surface left after a dissolving material has dissolved may have a roughness that is ordered and repeating.

In certain embodiments, the SMP polymer segments may be natural or synthetic, although synthetic polymers are preferred. The polymer segments may be non-biodegradable. Non-biodegradable polymers used for medical applications preferably do not include aromatic groups, other than those present in naturally occurring amino acids. The SMP utilized in the devices disclosed herein may be nonbiodegradable. In some implementations, it may be desirable to use biodegradable polymers in the SMP devices, such as when temporary stenting or occlusion is desired, for example.

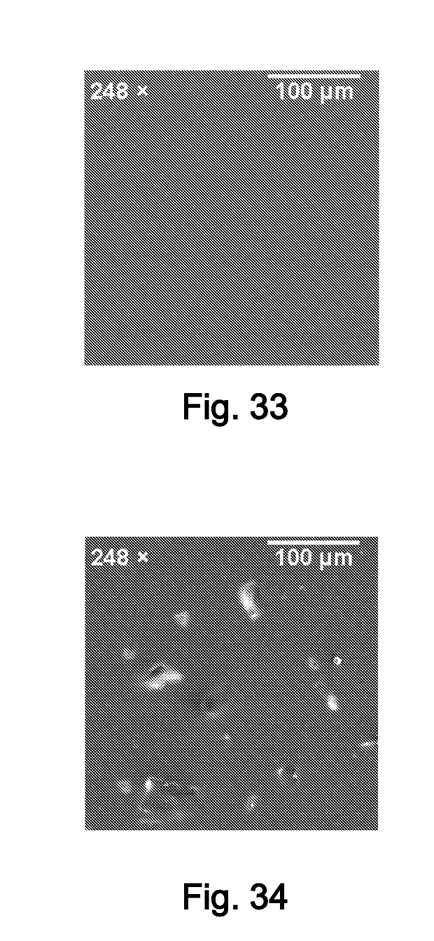

FIGS. 33 and 34 show the roughening of a surface of a SMP due to dissolving materials within and on the surface of the SMP. FIGS. 33 and 34 have been processed to show in black and white the surface variations that were in the original images as grey-scale variations. The images in FIGS. 33 and 34 were similarly processed from scanning electron microscope images taken at 248× resolution, and showing a legend bar that is 100 micrometers long to given dimension to the Figures. The scales of the images in FIGS. 33 and 34 are the same.

In FIG. 33, the dissolving material (particles of heparin, an anticoagulent drug), fills part of the body and surface of a SMP network. FIG. 33 shows a smooth surface comprising a SMP network and heparin particles. The scanning electron microscope used at the 248× resolution did not detect enough surface variation on the smooth combined surface to register significant grey scale variation.

FIG. 34 shows a significant increase in surface variation after heparin has been removed both from the combined surface of a SMP network and heparin, and from the body of the SMP network. After the heparin is removed (e.g., through dissolving), the SMP surface that is left contains significant surface variations. The surface variations are significant enough to obscure the resolution and length legend that appeared along the top of the image (similar to FIG. 33) before the image was processed. These surface variations may be used for different purposes. For example, purposes may include increasing biocompatability of the SMP in biological applications, or increasing surface area contact (over time, as a material leaves the SMP), thus affecting mechanical properties.

Dissolving materials, through their dissolution over time, may be used for many purposes. In one embodiment, the dissolution of a material may affect a dissolution or breaking up of a biomedical device over time. In another embodiment, the dissolution of a material may elute a drug, achieving a pharmacological purpose.

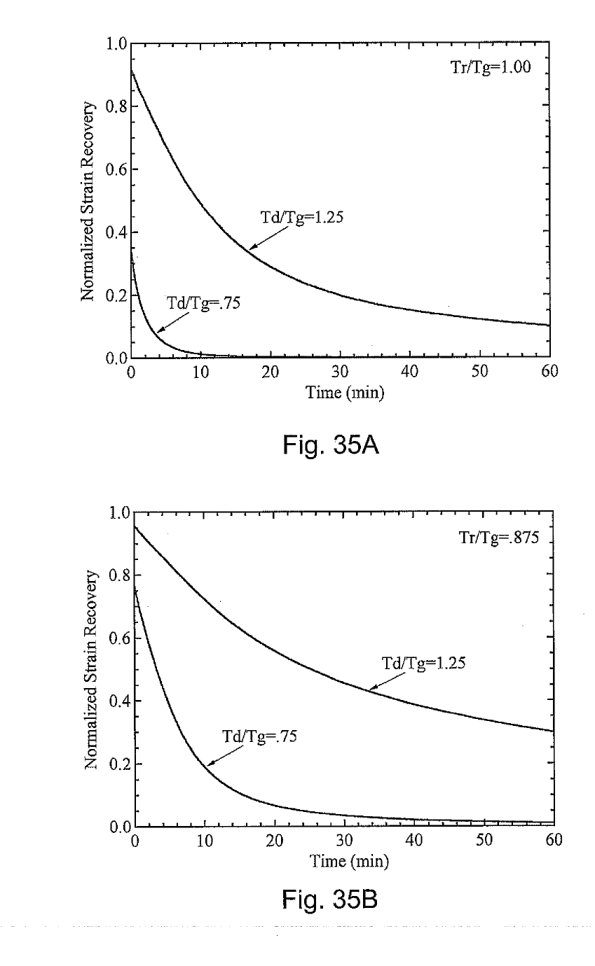

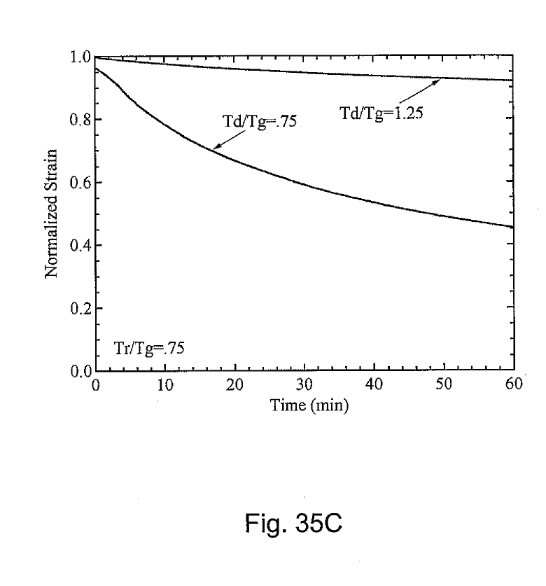

Deformation conditions can affect other properties of the SMP, for example thermomechanical manufacturing and handling processes can influence shape recovery. FIGS. 35A-35C show the effects of deformation temperature (T_{d}) on shape recovery. The stent with a higher T_{d} experienced a delay in recovery compared to its lower T_{d} counterpart.

Shape memory is driven by a favorable increase in entropy, thus lowering the free energy of the system. Sometimes, SMPs may be deformed at temperatures well above their glass transitions, thus requiring relatively little mechanical energy for deformation. When a polymer is deformed below its glass transition a significant amount of mechanical energy may be needed for deformation and the energy is stored in enthalpic internal energy wells. In this case, shape recovery is now driven by both a favorable increase in entropy and decrease in enthalpy, which may result in shape-memory activation at lower temperatures.

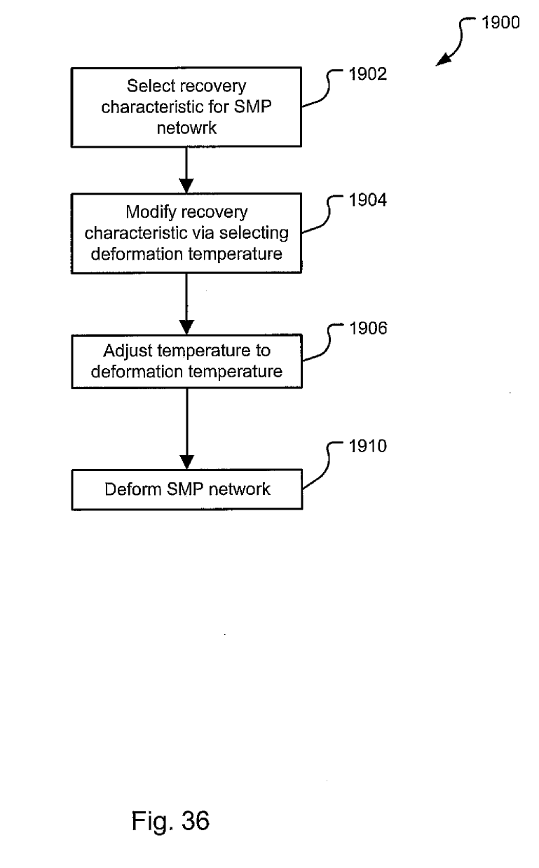

FIG. 36 shows a flow chart of an embodiment of a method 1900 for achieving a peak stress in a SMP during the recovery phase of the SMP via variations in the deformation temperature of the SMP during manufacturing. The embodiment of the method 1900 includes selecting a recovery characteristic 1902 of a SMP network. In one embodiment, the selecting operation 1902 may be performed by calculating or otherwise predicting the recovery characteristic of the SMP network. Modifying a recovery characteristic 1904 through selecting a deformation temperature of the shape memory polymer network may be performed based on the experimental results provided in and discussed with respect to FIGS. 35A-35C. Modifying a recovery characteristic 1904 may include modifying a manufacturing process of a SMP network to achieve a different recovery characteristic. In one embodiment, given a certain recovery environment, a recovery characteristic creates a recovery time that is not desirable, and the recovery time may be modified through modifying the recovery characteristic without changing the recovery environment. For example, with respect to FIG. 35B, a recovery time may be too long for recovering to 20% strain at a recovery temperature (Tᵣ)=0.875*T_{g}, based on a deformation temperature (T_{d})=0.75*T_{g}. A different T_{d} may be selected, for example, Td=1.25*T_{g}, to create a different recovery characteristic and therefore a different recovery time in the same recovery environment.

Selecting a deformation temperature and modifying a recovery characteristic thereby 1904 may use other deformation temperatures (T_{d}) than 0.75*T_{g} and 1.25*T_{g}. Curves such as those in FIGS. 35A-35C may be created for other scenarios and formulations and such curves are meant to serve as examples as well as substantive data. Modifying a recovery characteristic 1904 may also be performed based on other experimental results based on other SMP networks or other ranges of recovery temperatures, deformation temperatures, and/or transition temperatures as appropriate.

Method 1900 also includes causing the SMP network to be substantially at the deformation temperature 1906. Some techniques of causing the SMP network to be at a specified temperature are discussed elsewhere herein (e.g., temperature controlled liquid bath or contact with a heating or cooling element), and some are known to those with skill in the art. The causing operation 1906 may be performed to any degree of certainty, as appropriate. In one embodiment, contact with a heating or cooling element for a sufficient amount of time is an appropriate method both of causing a SMP network to achieve a deformation temperature 1906 and of assuring that the SMP network is substantially at the deformation temperature. In another embodiment, placement of a SMP network in a temperature controlled environment for a sufficient amount of time both causes the SMP network to attain the desired temperature 1906 and assures that the SMP network is substantially at the temperature (e.g., T_{d}).

The SMP network is deformed 1910 while at the deformation temperature. In one embodiment, the deformation temperature may be checked by any of the methods described above (e.g., with respect to causing the SMP network to be substantially at the deformation temperature 1906). In another embodiment, the deformation may be performed without continuing to control or to check the temperature of the SMP network.

As mentioned above, FIGS. 35A-35C are graphs of normalized strain versus time for different SMP networks. In other words, the graphs represent the recovery of stored strain in SMP networks as a function of time. In FIGS. 35A-35C, the SMP networks were formulated similarly, yet they exhibit different recovery processes and times based on the deformation temperatures and recovery temperatures to which the networks were exposed.

FIG. 35A is a graph of normalized strain versus time for a recovery temperature (T)=T_{g}. The graph shows recoveries of SMP networks deformed at T_{d}=0.75*T_{g} and T_{d}=1.25*T_{g}. As shown in the graph, the SMP network deformed at the lower temperature recovered more quickly in the same recovery environment.

FIG. 35B is a graph of normalized strain versus time for a recovery temperature, Tᵣ=0.875*T_{g}. The graph shows recoveries of SMP networks deformed at T_{d}=0.75*T_{g} and T_{d}=1.25*T_{g}. As shown in the graph, the SMP network deformed at the lower temperature recovered more quickly in the same recovery environment. Comparing the graphs in FIG. 35B to those in FIG. 35A, the recovery process of the SMP network is also affected by the lowering of the recovery temperature, though recovery may still be completed for recovery temperatures below T_{g}.

FIG. 35C is a graph of normalized strain versus time for a recovery temperature, Tᵣ=0.75*T_{g}. The graph shows recoveries of SMP networks deformed at T_{d}=0.75*T_{g} and T_{d}=1.25*T_{g}. As shown in the graph, the SMP network deformed at the lower temperature recovered more quickly in the same recovery environment. Comparing the graphs in FIG. 35C to those in FIG. 35B, the recovery process of the SMP network is also affected by the lowering of the recovery temperature, though recovery may still be completed for recovery temperatures below T_{g}.

### SMP Stent and Plug Devices

The SMP stent and plug devices disclosed herein comprise SMPs as described in detail above and may comprise any of the above described polymers and/or properties. The SMP stents and plugs described herein below may be referred to simply as “stents” or “plugs” for brevity but it should be understood that all of the stents and plugs comprise SMPs. As also described above, any of the below SMP stents and plugs may further be infused or eluded with drugs and/or radiopaque materials.

The SMP stents and plugs disclosed here have a number of unique characteristics due to the incorporation of the SMPs. The SMP stents and plugs have the ability to be highly compacted for delivery yet may expand to accommodate large, and/or non-standard anatomical geometries. The thermomechanical properties of the SMPs offer a greater level of customizability than standard metal-based and coated stents and plugs. Customizability includes, for example, the ability to tailor mechanical properties such as rubbery modulus, deployment time, and device conformability, as explained in detail above. Additionally, any of the SMP stents and plugs disclosed herein may be formed from, or incorporate in various percentages, a hydrogel material to increase the size or thickness of the SMP stent or plug post-implantation (i.e., upon absorbing fluid).

The SMP stents and plugs disclosed herein may be delivered and/or retrieved using catheter approaches. The SMP material allows the stents/plugs to be compacted for delivery and/or retrieval, while still providing a stent/plug with the appropriate anatomical or vessel size once expanded. The simplest way to deliver the SMP stent/plug may be to simply compact the stent/plug to its smaller size and then insert the stent/plug into a catheter for delivery. The catheter would then be inserted into a patient and the stent/plug would be pushed out of the catheter at the desired location and exposed to stimuli to expand the stent/plug in the desired location. Conversely, the stent/plug may be removed by compacting (such as by exposure to stimuli) and then by grasping the stent/plug and pulling the stent/plug into a catheter and out of the body.

SMP stents and plugs may also be removed by several other methods including localized heating or cooling. Heating may soften the SMP stent/plug for easier removal. Cooling may compact the SMP stent/plug, such as by inactivation of the SMPs, for easier and less invasive (due to narrower diameter) removal. In some implementations, such as after cooling, if the stent/plug is compressed, the SMP stent/plug may be drawn back into the catheter and then drawn out of the body. Heating of the SMP stent/plug may be accomplished by injecting sterile saline of a temperature higher than body temperature in the vicinity of the stent/plug. Similarly, cooling of the SMP stent/plug may be accomplished by injecting sterile saline of a temperature lower than body temperature in the vicinity of the stent/plug.

In some embodiments a SMP stent/plug may also incorporate heating or cooling elements within its body, which may be activated by connecting a device, such as catheter, to a heating or cooling device to generate a small electrical charge and change the temperature of the SMPs to soften the material or inactivate the SMPs. These heating or cooling elements may comprise an electrically conductive element, such as a wire or several wires. Other heating, cooling and removal techniques may be utilized herein to remove the SMP stent/plug. As also described above, the SMP stents/plugs may be dissolvable and/or biodegradable, and thus would not need to be removed.

The SMP stents and plugs described below may further be designed to incorporate radiopaque materials. In some embodiments the various radiopaque particles or strips may be infused into the polymers of the SMP stents and plugs to facilitate location of the SMP stents and plugs once deployed within a patient. These SMP stents and plugs having radiopaque particles therein may be easily located within a patient simply by using x-ray, MRI and/or ultrasound imaging. Exemplary radiopaque particles may include gold powder, thin strips of gold foil, tungsten powder, gold-tantalum and iron-oxide, and iodipamide, and/or a radiopaque contrast medium.

The SMP stent and plugs may be manufactured by several methods including, for example, injection molding or blow molding. In one exemplary method, a SMP stent or plug may be manufactured by injecting a liquid monomer formulation into an appropriate glass mould and photopolymerizing. Glass tube moulds may be blown to match specific geometrical parameters and allow for the SMP stents and plugs to be patient size- and shape-specific if needed. Once the polymer is cured, the glass mould may be gently broken and the SMP stent or plug may be removed. The SMP stent or plug may then be compacted through an extrusion die at room temperature and cooled in a freezer to lock in this temporary packaged state. The SMP stent or plug may then be removed from the die and placed in a catheter for implantation.

### Solid and Fenestrated SMP Stents

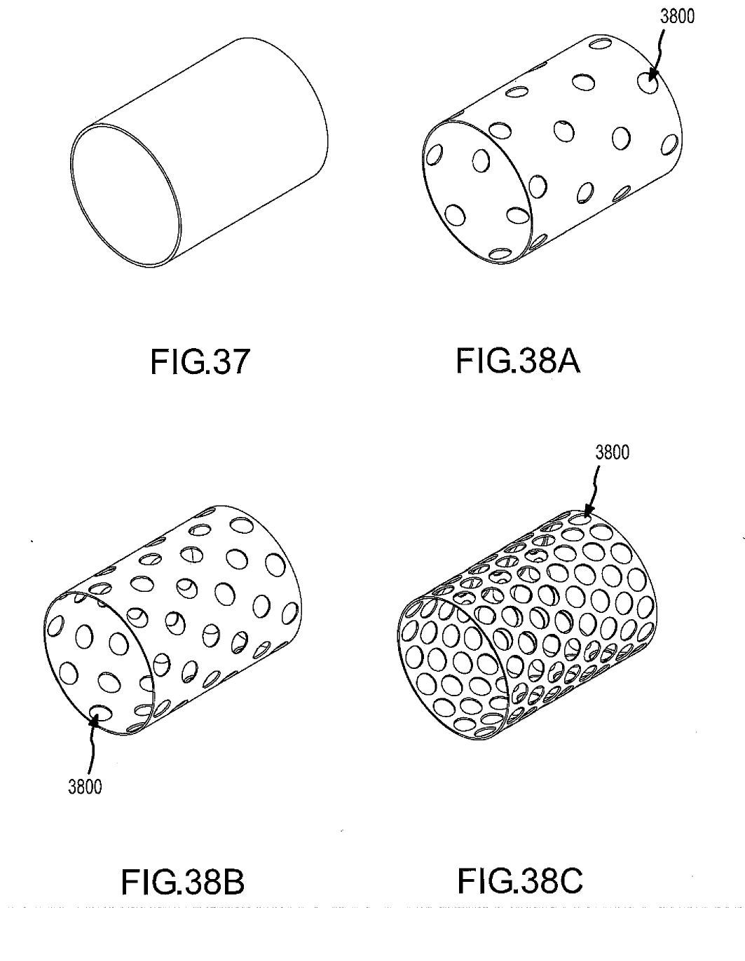

SMP stents may be formed in hollow cylindrical tubular shapes, as shown in FIG. 37. SMP stent geometry may vary from a solid SMP stent without holes or openings, as shown in FIG. 37, to fenestrated SMP stents having holes or openings, as shown in FIGS. 38A-38C. Solid or nonfenestrated SMP stents, as shown in FIG. 37, may be advantageous in applications where a self-expanding, coated SMP stent is to be utilized, such as for treatment of abdominal aortic aneurysms (AAA) or for endovascular stent grafts, for example. In some embodiments these SMP stents may have one or both ends flared down or up to produce ends that are smaller or larger in diameter than the diameter of the middle of the stent. Because the solid stent has a larger surface area than that of the fenestrated stents, the solid stents may provide a larger surface coating area for drug elution and more rigid support for maintaining vessel diameter. These solid SMP stents have been manufactured and tested and they conformed well to the lumen of a tube without buckling, slipping, sliding or exhibiting any other undesirable shape deformation.

The fenestrated SMP stents may be advantageous in applications where coated stents are not utilized and where some type of internal flexible scaffold can change with vessel pulsation. Fenestrated SMP stents have portions of wall material removed to form a flexible support structure of scaffold, which may be more flexible than solid SMP stents. Fenestrated SMP stents may be utilized as venous stents or as SFA stents, for example. Because the fenestrated SMP stents have some wall material removed they have less surface area and therefore, may be more flexible and/or less rigid than solid SMP stents. The fenestrated SMP stents have the ability to move or flex with vessel pulsation, making them advantageous for use as venous stents.

In one embodiment, SMP stent geometry may be varied by removing circular portions to create circular holes or openings 3800, forming the fenestrated SMP stents shown in FIGS. 38A-38C. In other embodiments, fenestrated SMP stent geometry may be varied by removing other wall portions or shapes from the fenestrated SMP stent, such as by removing strips, slits, diamond-shapes, square-shapes, etc. In some embodiments, the wall portions removed from the fenestrated SMP stent may comprise circumferentially or longitudinally oriented slits. The shapes, sizes, and arrangement of the wall material removed herein are exemplary only and the shapes, sizes, and arrangement of the wall material removed may vary significantly depending upon the final application of the fenestrated SMP stent.

Different percentages of wall material may be removed from the fenestrated SMP stent to tailor the fenestrated SMP stent properties to specific applications, such as amount of modulus or flex of the fenestrated SMP stent. The amount of wall material removed may vary from approximately 10% to approximately 50%, as shown in FIGS. 38A, 38B and 38C. As shown in FIG. 38A, in some embodiments it may be desirable to remove only a small portion of material, such as approximately 10% to provide a fenestrated SMP stent with less flex and more rigid support. As shown in FIG. 38B, it may be desirable to remove a larger portion of material, such as approximately 30% to provide a more even balance between the flex and rigid properties. As shown in FIG. 38C, it may be desirable to remove a significant portion of material, such as approximately 50%, to provide a fenestrated SMP stent with significant flex and minimal rigidity. The percentages of material removed herein are exemplary only and the amount and/or percentage of materials removed may vary significantly depending upon the final application.

### Slit SMP Stents

In one embodiment a cylindrical SMP stent may further comprise a longitudinal slit. The SMP slit stent may be formed by creating a longitudinal slit extending the length of the stent, such that two adjacent slit edges 3902, 3904 are created. The slit edges 3902, 3904 of the stent may overlap each other, allowing the slit stent to be rolled up many times for more compact delivery. The SMP slit stent may be formed from a single strip or sheet of SMP material or from a tube of SMP material. In some implementations, the slit stent edges 3902, 3904 may be thicker than the remaining body portion of the SMP slit stent.

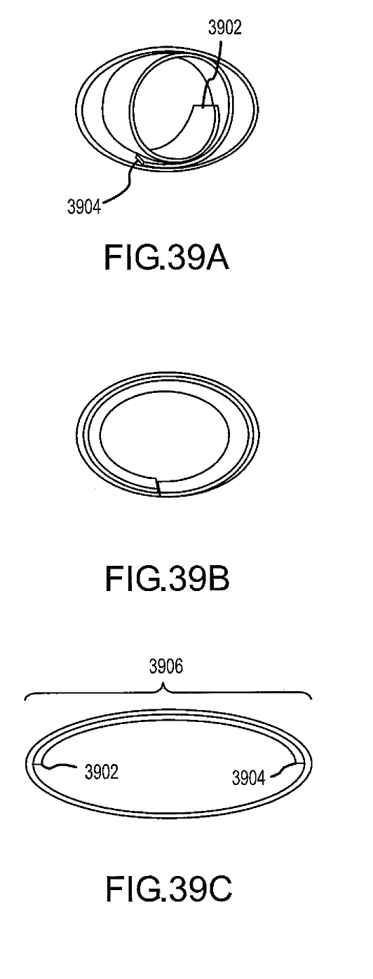

When compacted, the slit stent edges 3902, 3904 may overlap one another multiple times forming a rolled cylinder, as shown in FIG. 39A. As the SMP slit stent begins to expand or recover its original shape when exposed to stimuli, the SMP slit stent begins to unroll, as shown in FIG. 39B. Even when fully expanded or recovered, the slit edges 3902, 3904 of the SMP slit stent may still overlap one another, as shown in FIG. 39C. The overlapping portion 3906 is formed by the overlap of the slit edges 3902, 3904. The overlapping portion 3906 provides greater expansion capacity. The SMP slit stent may have the ability to continually expand or grow with a vessel over a longer period of time to achieve a greater diameter within a vessel. This may be particularly advantageous for growth of the SMP slit stent with a vessel and for pediatric applications.

The longitudinal slit in the SMP slit stent may also minimize stresses on the SMP slit stent as the vessel expands and contracts. The overlapping edge portions 3902, 3904 provide additional expansions area for the SMP slit stent to be more easily expanded and compacted. As the SMP slit stent expands, the overlapping edge portions 3902, 3904 may simply slide over one another to create a smaller overlap portion 3906. As the SMP slit stent compacts, the overlapping edge portions 3902, 3904 may simply slide over one another to create a larger overlap portion 3906. The ability of the overlapping edges portions 3902, 3904 to smoothly and easily slide over one another minimizes the stresses on both the SMP slit stent and on the vessel walls as expansion and contraction (or vessel pulsation) occur.

Additionally, the ability of the SMP slit stent to cover a wide range of vessel size diameters provides a wider range of applications for the SMP slit stent. The SMP slit stent may also enhance deployment time of the SMP slit stent, which minimizes risk to patients during delivery. Furthermore, the SMP slit stent may reduce the internal stresses on the stent itself and may increase the compliance of the SMP slit stent, while still allowing the SMP slit stent to grow with the vessel over time. These slit SMPs stents have been manufactured and tested and they conformed well to the lumen of a tube without buckling, slipping, sliding or exhibiting any other undesirable shape deformation.

### Coiled SMP Stents

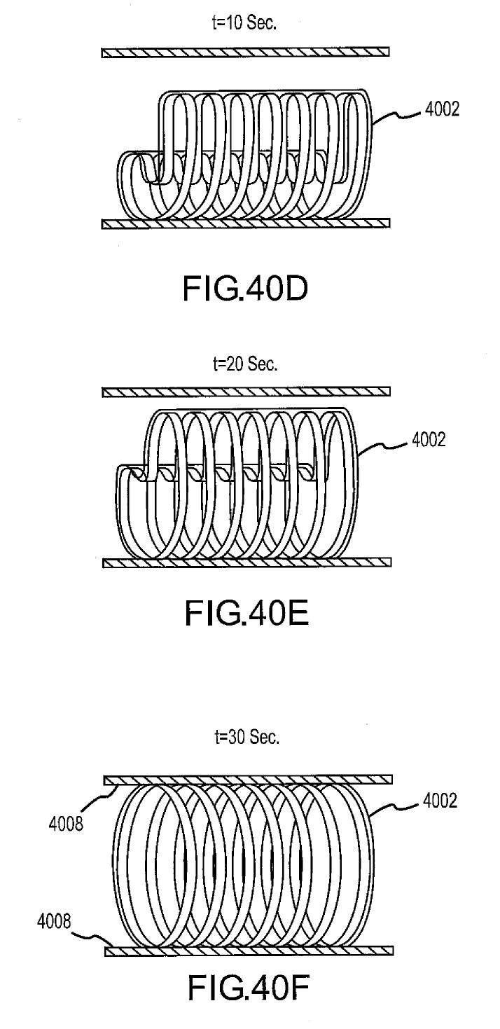

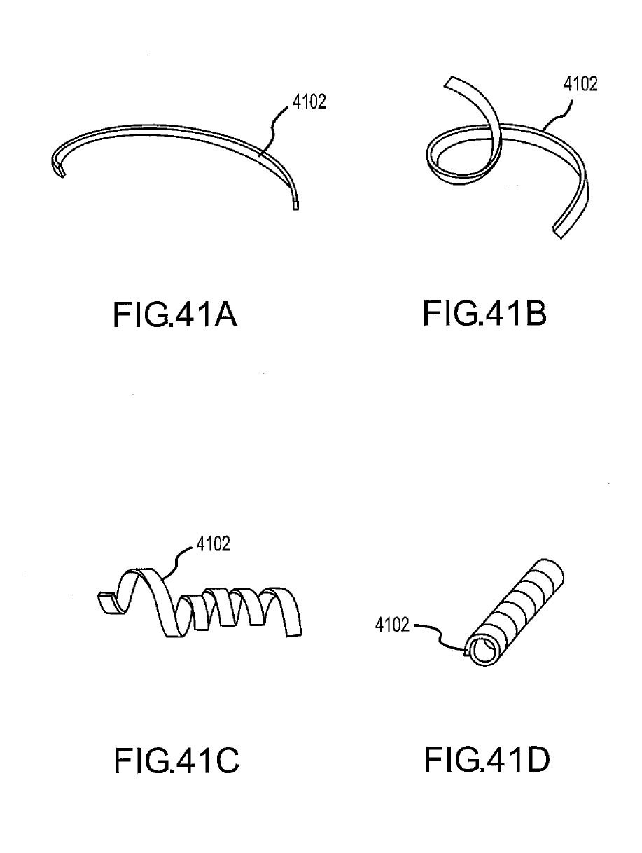

In one embodiment a coiled SMP stent may be formed from a strip of SMP material, as shown in FIGS. 41A-41D. The elongated strip of SMP material 4102 (shown in FIG. 41A) may be formed with shape memory properties that cause the strip of material 4102 to begin to curl when exposed to stimuli, as shown in FIG. 41B. The strip of SMP material 4102 may continue to curl tighter, as shown in FIG. 41C until it forms a tightly wound coil 4104, as shown in FIG. 41D.

Because the outer wall of the coiled SMP stent is formed of individual coils, rather than a solid continuous sheet of material, the coiled SMP stent may exhibit high flex while still having some rigidity. The high flex of the coiled SMP stent may be advantageous for use in vessels where pulsation (i.e., expansion and contraction occur). The ability of the coiled SMP stent to flex with pulsation minimizes stresses on the vessel and on the coiled SMP stent. The coiled SMP stent may also be advantageous for stenting vessels which have a bend or curve, as the coils may move somewhat independently from one another to bend in different directions.

### Insertion and Deployment of SMP Stents

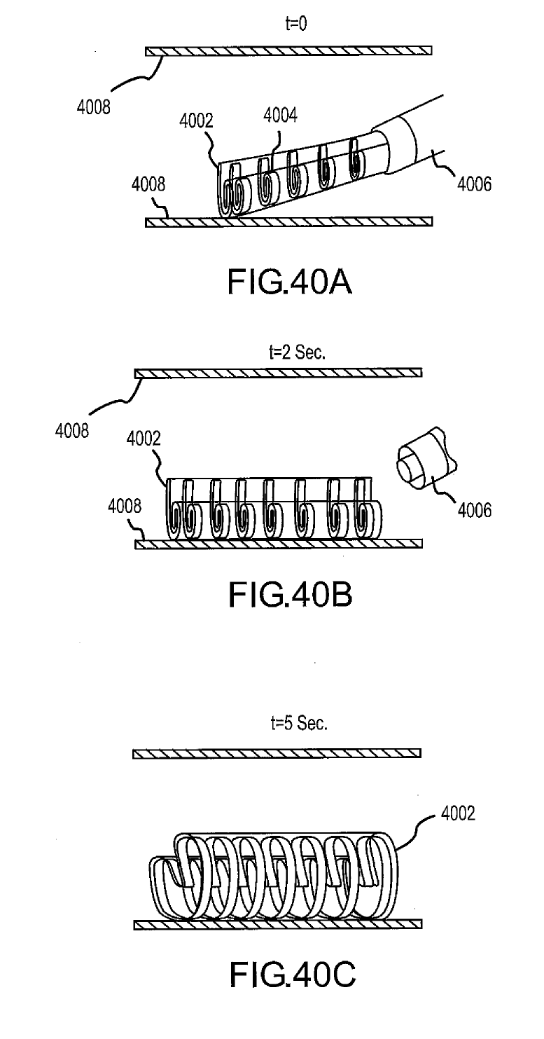

The SMP stents described herein may all be deployed by a catheter in a compacted form. The compacted SMP stents may be loaded into a catheter and the catheter may be inserted into the patient, such as into a cardiovascular vessel, for example. Once the catheter reaches the desired stent implant location, the stent may be pushed out of the catheter and into the desired location, such as into a cardiovascular vessel, for example. Once the stent has been position properly within the vessel it may then be exposed to stimuli, such as heat, to induce expansion or recovery of its memory shape. The SMP stent may then expand to fill the lumen of the vessel, providing structural support to maintain a desired diameter within the vessel. Various examples of SMP stent insertion and deployment are shown in FIGS. 40A-40F, 42A-42D, and 43A-43E.

FIGS. 40A-40F illustrate exemplary deployment of a solid SMP stent into the lumen of a vessel. FIG. 40A illustrates the deployment of a solid SMP stent 4002 having circumferential ribs 4004. The circumferential ribs 4004 may be formed of a radiopaque material to clearly illustrate the expansion of the solid SMP stent 4002 as a function of time. FIG. 40A illustrates the solid SMP stent 4002 in a compacted form as it is being pushed out of a catheter 4006 and into the lumen of vessel 4008 at initial time t=0. FIG. 40B illustrates the solid SMP stent 4002 as it completely expelled from the catheter 4006 and has settled into place in the lumen of the vessel 4008 and has begun to expand at time t=2 seconds. FIG. 40C illustrates the solid SMP stent 4002 as it continues to expand at time t=5 seconds. FIG. 40D illustrates the solid SMP stent 4002 as it continues to expand at time t=10 seconds. FIG. 40E illustrates the solid SMP stent 4002 as it continues to expand at time t=20 seconds. FIG. 40F illustrates the solid SMP stent 4002 as it reaches full expansion and contacts the lumen of the vessel at time t=30 seconds.

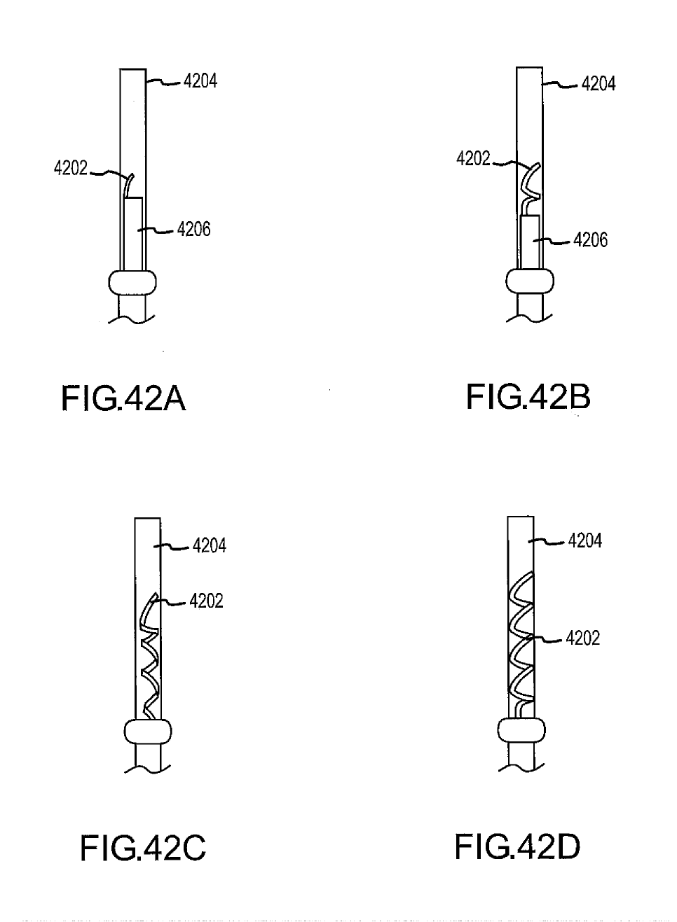

FIGS. 42A-42D illustrate exemplary deployment of a SMP coiled stent 4202 within a tube 4204. FIG. 42A illustrates the end portion of a SMP coiled stent 4202 as it has begun to be pushed out of a catheter 4206 which has been inserted into the clear tube 4204. The tube 4204 shown herein is exemplary only for clarity of illustration and may represent the lumen of a vessel, such as a cardiovascular vessel, for example. FIG. 42B illustrates more of the SMP coiled stent 4202 as it continues to be pushed out of the catheter 4206 and into the clear tube 4204. FIG. 42C illustrates the entire SMP coiled stent 4202 as it has been pushed completely out of the catheter 4206 and into the tube 4204 (the catheter 4206 has been removed from the tube) and begins to expand. FIG. 42D illustrates the SMP coiled stent 4202 as it reaches its fully expanded shape and contacts the outside walls of the clear tube 4204.

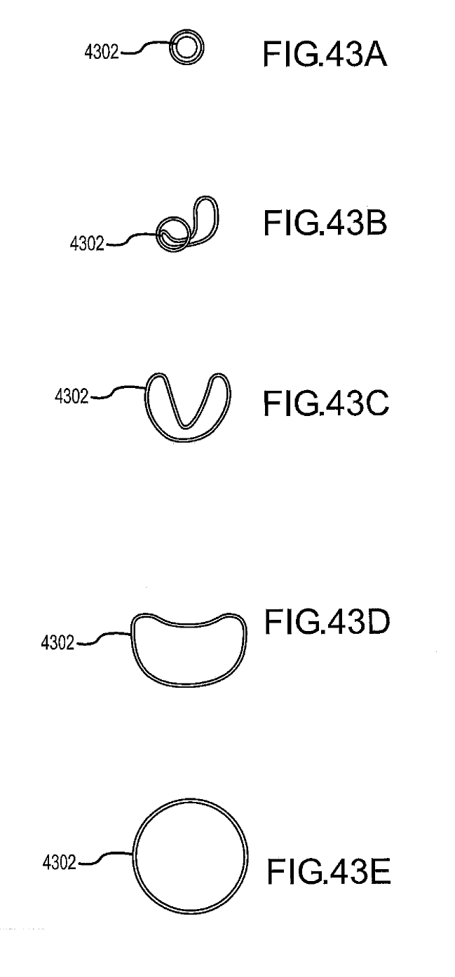

FIGS. 43A-43E illustrate exemplary cross-sectional deployment of a compacted or rolled SMP stent 4302 which has been compacted for delivery. The SMP stent 4302 shown herein may be a solid SMP stent or a fenestrated SMP stent. FIG. 43A illustrates the fully compacted SMP stent 4302 which has been rolled onto itself to be as compact as possible for delivery. FIG. 43B illustrates the SMP stent 4302 after it has been exposed to stimuli and begins to unroll and expand. FIG. 43C illustrates the SMP stent 4302 as it continues to unroll and expand. FIG. 43D illustrates the SMP stent 4302 as it continues to unroll and expand. FIG. 43E illustrates the SMP stent 4302 as it reaches its fully expanded cylindrical shape.

### SMP Plug Devices

The SMP plugs described herein may function as SMP occluding devices (or plugs) to close or plug vascular holes or defects, close or plug the lumen of a vessel or other cavity, and/or plug septal defects in the heart, for example. These SMP plugs may be formed in a number of different design variations. In some embodiments the SMP plugs may be designed to be solid, hollow, or some combination thereof.

In some embodiments, the hollow pockets in the SMP plugs may be filled with a solution or other material, such as saline solution, water, air, or other filler material. The use of additional materials within the hollow pockets in the SMP plugs may help to structurally reinforce the plugs described herein and may help them remain in their fully expanded state (once expanded). In other embodiments the hollow portion of the SMP plugs may be filled with a material that can be dissolved and/or removed at a later time, such as one or more dissolvable polymers, gels, water or saline solution, or air.

The SMP plugs may also be formed to incorporate medications therein. In some embodiments the SMP plugs may be coated with a medication. In other embodiments the SMP plugs may comprise a pocket filled with medication which may be dissolved and released upon deployment or at a later time. In yet additional embodiments the SMP plugs may be designed to incorporate hydrogel materials. The hydrogel material may enhance swelling of the SMP plug as it expands to provide additional growth of the SMP plug, resulting in a larger SMP plug and a more effective liquid-tight seal. The incorporation of a hydrogel material will be described in more detail below with reference to FIGS. 53 and 54.

### Bulb-Shaped SMP Plugs

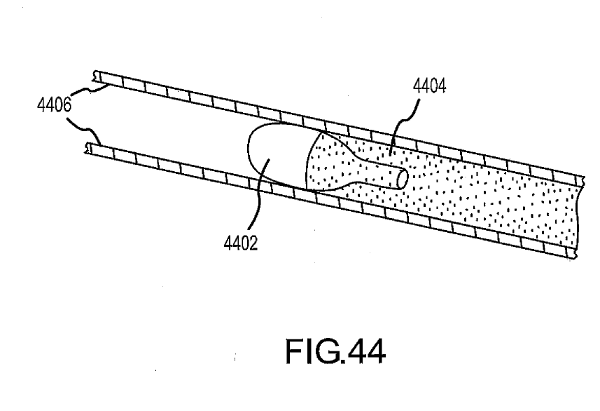

A SMP plug device may be formed in the shape of a bulb 4402 or plug, when activated or expanded, as shown in FIG. 44. The design of the SMP plug may be that of a bulb-like shape meant to block the passageways or lumens of vessels to form a liquid-tight seal or plug an opening, such as for treatment of a septal defect. The bulb-like shape may be an approximately oval-shaped device 4402 (shown in FIG. 44) and may be referred to as a SMP plug or bulb design 4402 herein.

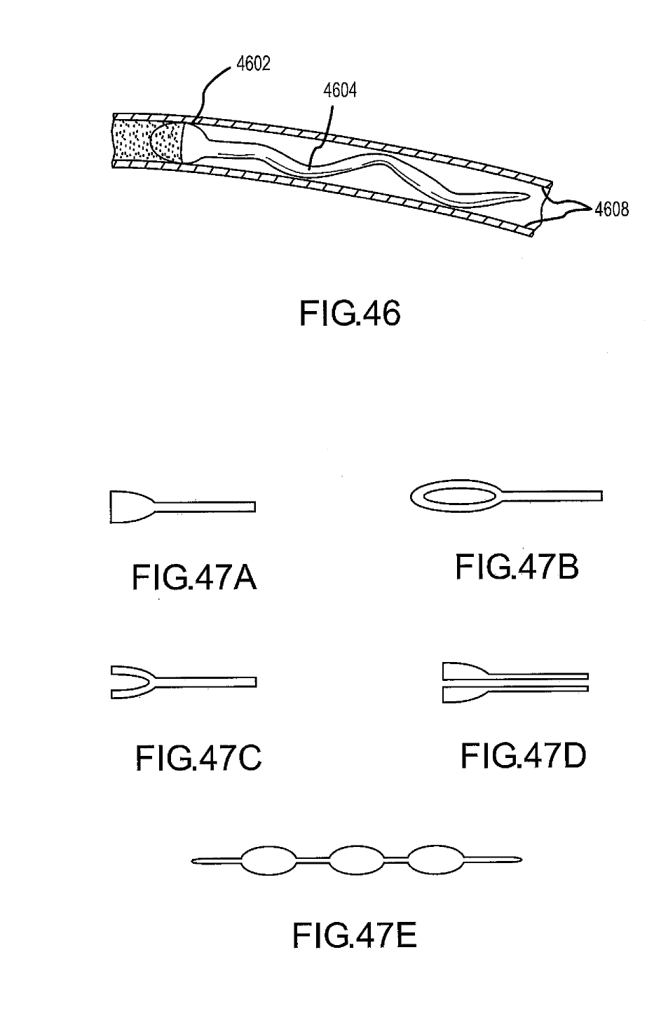

In some embodiments the bulb-shaped SMP plug 4402 may further comprise one or more end portions 4404, shown in FIG. 44, which may make it easier to grasp and guide the SMP plug 4402 for better control during delivery and/or removal. In other embodiments, such as that shown in FIG. 46, the bulb-shaped SMP plug 4602 may further comprise a more elongated tail portion 4604. This elongated tail portion 4604 may be formed to be more lengthy to prevent slippage or movement of the bulb-shaped SMP plug 4602 within the lumen of the vessel 4608 and to facilitate grasping and guiding of the bulb-shaped SMP plug 4602 during delivery and/or removal.

FIG. 44 illustrates an exemplary bulb-shaped plug 4402 deployed within a lumen of a vessel 4406 and forming a liquid-tight seal within the lumen of the vessel 4406. In some implementations, multiple bulb-shaped SMP plugs may be used to help ensure full blockage of the vessel or lumen or full occlusion of an opening or septal defect. In some implementations a single bulb-shaped SMP plug may comprise one or more bulb-shaped or plug-shaped portions on one device, as shown in FIG. 47E.

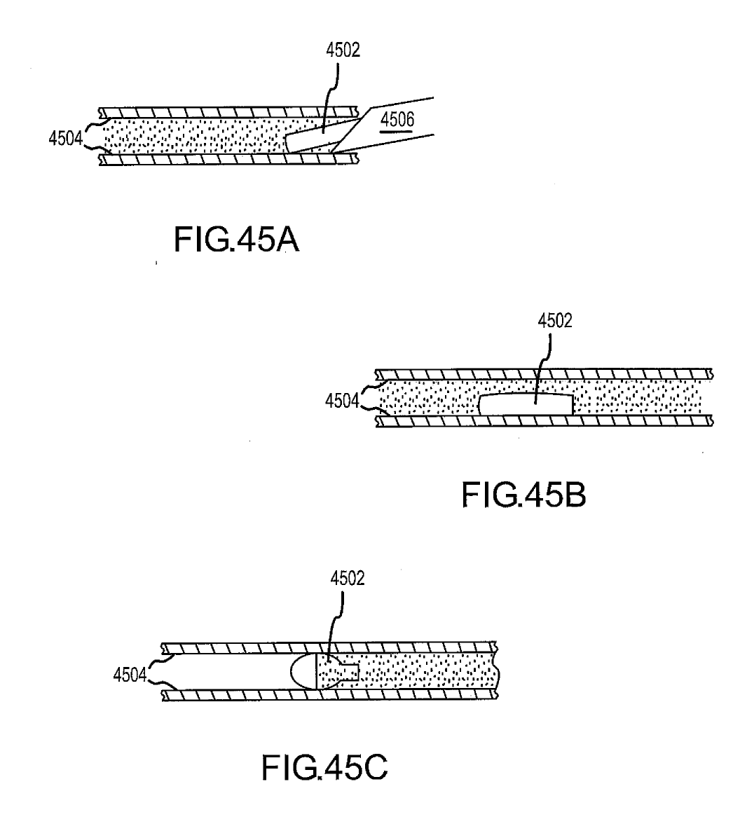

FIGS. 45A-45C illustrate exemplary deployment and expansion of a bulb-shaped SMP plug 4502 within a lumen of a vessel 4504. FIG. 45A illustrates a fully compacted bulb-shaped SMP plug 4502 as it is pushed out of a catheter 4506 and into the lumen of a fluid-filled vessel 4504. FIG. 45B illustrates the bulb-shaped SMP plug 4502 (expelled from the catheter 4506) as it settles into place within the lumen of a fluid-filled vessel 4504 and as it begins to expand (after exposure to stimuli). FIG. 45C illustrates the bulb-shaped SMP plug 4502 as it reaches its fully expanded state and contacts the interior wall of the fluid-filled vessel 4504, forming a fluid-tight seal within the lumen of the vessel 4504.

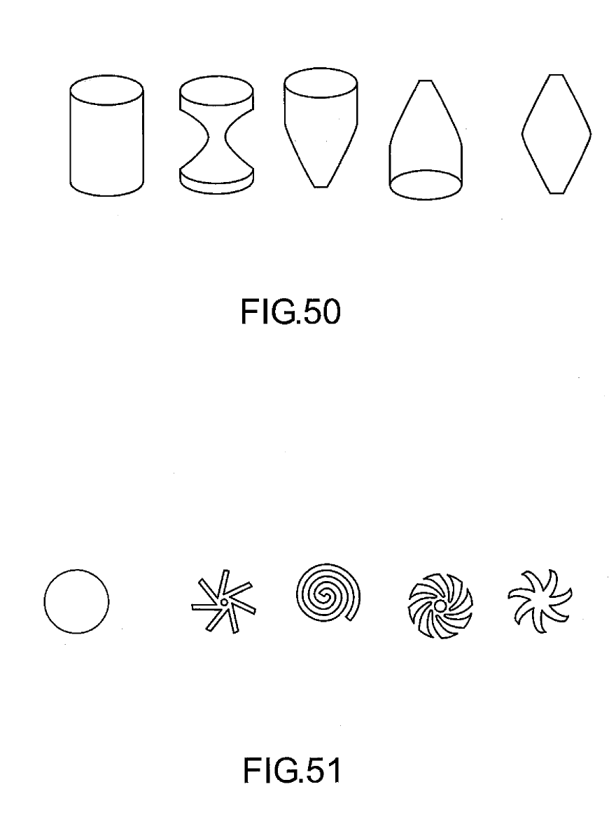

SMP plugs for occluding vessels, tube, cavities, and/or plugging septal defects may be formed in a variety of shapes and sizes and configurations. FIGS. 47A-47D, 48A, 48B, 50 and 51 all illustrate a number of different exemplary designs for SMP plugs. FIG. 50 illustrates a number of different exemplary shapes for solid SMP plugs. The solid SMP plugs shown in FIG. 50 may be utilized to form a fluid-tight seal, completely blocking the fluid passage through the lumen of a vessel. In addition to solid SMP plugs, a variety of other partially-solid SMP plugs may be utilized. FIG. 51 illustrates a number of different exemplary cross-sectional shapes for SMP plugs useful as vascular defect closure plugs. These SMP plugs may a solid central body portion with several leg portions radiating or extending therefrom to help reduce or regulate the flow of fluid through the lumen of a vessel without completely blocking the fluid flow.

Additional exemplary SMP plug designs are illustrated in FIGS. 47A-47D. FIG. 47A illustrates an exemplary half-oval-shaped solid SMP plug. FIG. 47B illustrates an exemplary oval-shaped hollow SMP plug. FIG. 47C illustrates an exemplary half-oval-shaped hollow SMP plug. FIG. 47D illustrates an exemplary half-oval-shaped SMP plug having a hollow central shaft. The hollow portions of the SMP plugs may be filled with fluid or other filler materials, such as saline solution, water, air, dissolvable polymer, or gel as previously described above. The sizes and shapes of the SMP plugs described herein are exemplary only and the sizes and shapes may vary significantly depending upon the final application.

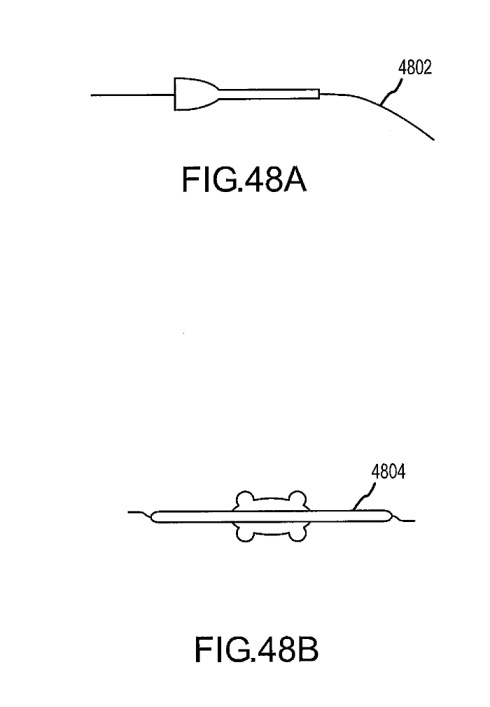

FIGS. 48A and 48 B illustrate SMP plugs which may further include a guidewire 4802 or other central shaft portion 4804. This guidewire 4804 or other central shaft portion 4804 may be formed of a metallic material, such as Nitinol®, and may extend through the length of the SMP plug. The guidewire 4802 or other central shaft portion 4804 may be used to help control or guide the placement of the SMP plug within the patient. The guidewire 4802 or other central shaft portion 4804 may also be used for pushing the SMP plug out of a catheter during delivery and/or for grasping the SMP plug and pulling it into the catheter during retrieval. Once the SMP plug is in proper position within the patient, the guidewire 4802 or other central shaft portion 4804 may be removed. Alternatively, in some embodiments the guidewire 4802 or other central shaft portion 4804 may be permanent and remain with the SMP plug. In some embodiments the guidewire 4802 or other central shaft portion 4804 may provide additional structural support to the SMP plugs and/or may enhance radiopacity of the SMP plugs.

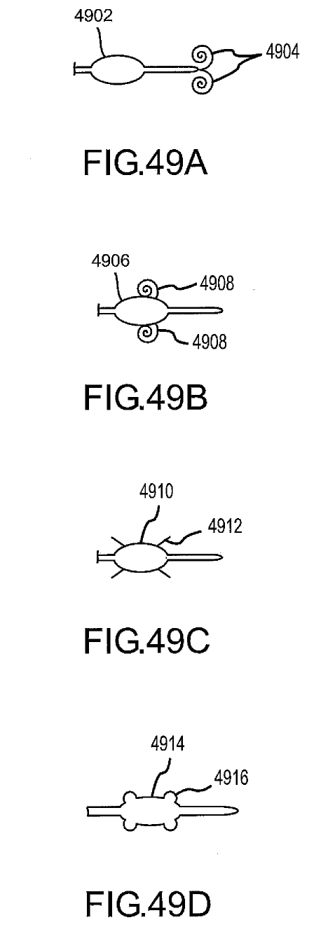

Several additional adjustments may be made to the design of the SMP plugs to enhance their functionality. In some implementations, SMP plugs may also be designed to incorporate securing mechanisms, such as hooks, anchors, barbs, or other protrusions to securely hold the SMP plugs in place once positioned. FIGS. 49A-49D illustrate exemplary embodiments of bulb-shaped SMP plugs incorporating hooks, anchors, and barbs in a variety of different configurations. FIG. 49A illustrates an exemplary embodiment of a SMP plug 4902 having hooks 4904 positioned on an elongated end portion of the SMP plug 4902. FIG. 49B illustrates an exemplary embodiment of a SMP plug 4906 having hooks 4908 position on the body portion of the SMP plug 4906. FIG. 49C illustrates an exemplary embodiment of a SMP plug 4910 having barbs 4912 positioned on the body portion of the SMP plug 4912. FIG. 49D illustrates an exemplary embodiment of a SMP plug 4914 having anchors 4916 positioned on the main body portion of the SMP plug 4914. It should be understood that the SMP plugs described herein may incorporate one or many of any of the hooks, barbs, or anchors in any variety of combinations. Similarly, these hooks, barbs, or anchors may be used on SMP plugs having any shape or size.

The barbs, hooks, anchors, or other protrusions may be formed in any size and may be added to the SMP plug to help secure or anchor the SMP plugs within the lumen of a cardiovascular vessel or within a septum. In some embodiments the barbs, hooks, or anchors may be positioned on the bulb-shaped body of the SMP plugs, as shown in FIGS. 49B-49D. In other embodiments, the barbs, hooks, or anchors may be positioned on an elongated end portion of the SMP plug, away from the bulb-shaped body portion, as shown in FIG. 49A. In this embodiment the hook or anchor may be hooked onto or anchored on an end of vessel or other structural element to hold the SMP plug in place. The distance between the hooks/barbs/anchors and the bulb-shaped body portion may vary over a wide range, such as from 0.1 mm to 40 mm. The hooks/anchors/barbs may be positioned on either end of the SMP plugs. These implementations may help prevent or minimize any movement of the SMP plugs within a vessel, enhancing the secure fit within the lumen of the vessel and increasing the effectiveness of the SMP plug.

### Coiled SMP Plugs

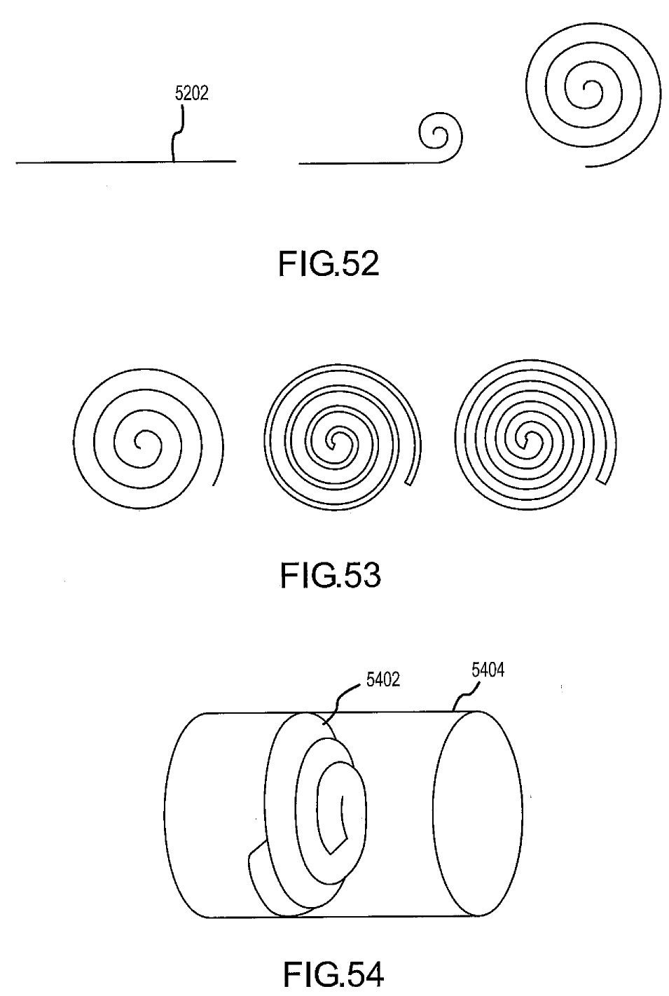

FIGS. 52, 53 and 54 illustrated coiled SMP plug devices. As shown in FIG. 52, the coiled SMP plug device may be formed of an elongated strip or sheet of SMP material 5202. Once exposed to stimuli, the coiled SMP plug will continue to coil onto itself until a tightly wound coil shaped SMP plug has formed (shown at right in FIG. 52). The coiled SMP plug may also be used to block or occlude a vessel or cardiac defect. In additional embodiments, the SMP plugs may incorporate hydrogel materials so that the SMP plugs swell and increase thickness as well as coiling.

FIG. 53 illustrates the coiled SMP plug before swelling (at left) and then shows the gradual swelling of the hydrogel material (at right) over time. The incorporation of the hydrogel material increases the thickness of the coiled SMP plug, helping to more completely block the vessel, resulting in a more effective plug. FIG. 54 illustrates a fully swollen and expanded hydrogel coiled SMP plug 5402 occluding a clear tube 5404.

Advantages of a coiled SMP plug design include the use of a small delivery catheter and gentle self-expansion of the SMP plug. Before insertion, the coiled SMP plug may be uncoiled to a straight shape (shown in FIG. 52 at left) that fits easily into a catheter, which may then be inserted into a cardiovascular vessel. Once inserted into the cardiovascular vessel and free of the catheter, the body's natural heat may activate the shape-memory effect and return the coiled SMP plug to its original coiled shape, effectively occluding the cardiovascular vessel, as shown in FIG. 54. Coil shapes have been manufactured to be compacted into catheters with internal diameters ranging from 0.9 to 2.0 mm, and expand into tubes of diameters ranging from 1.5 to 4.0 mm.

The coiled SMP plugs may require some fibrous growth to completely block the vessel. Thus, the coiled SMP plugs may also incorporate fibrous structures or mesh in areas to encourage additional fibrous growth over time. Because the coiled SMP plugs may have delayed occlusion effectiveness, it may be desirable to use the coiled SMP plugs in combination with the bulb-shaped SMP plugs to provide immediate occlusion. In some situations, the coiled SMP plugs may accumulate significant fibrous growth over time, resulting in a permanent and effective occluding device (i.e., plug).

In other embodiments, the coiled plugs may incorporate barbs, hooks, or anchors, and/or may be used in combination with bulb-shaped SMP plugs and/or stents as described above. The coiled SMP plugs may also incorporate radiopaque materials, medications, and/or be co-polymerized with thin strings or guidewires, for example, made of fabric, metal, or other polymers, to increase tensile strength, also as described above. It should be further understood that the SMP plugs described herein may incorporate any number of the above-described features in a variety of different sizes, shapes, and configurations.

### Methods of Manufacturing SMP Stents and Plugs (Including Examples)

Several methods may be used to manufacture the SMP stents disclosed herein. One exemplary process involves fabricating the stents and plugs by photopolymerizing them in a mould and then machining them or laser cutting them to a final state. A Teflon rod may be machined to size and fitted with a glass tube. The gap between the rod and the glass tube may then be filled with a monomer solution. The Teflon-tube mold may then be rotated and exposed to UV light to photopolymerize the monomer solution. After about 10 minutes, the glass tube and polymer may be separated from the Teflon rod and cured at 90° C. for 1 hour to ensure complete conversion of the monomers into the polymer. The polymer, now in the form a tube is then removed from the glass tube and further machined. The solid SMP tube can be cut to length, lathed, and further machined (e.g., by computer numerical control (CNC)) on a mandrel to create the final stent design. The final stent design may be a porous or slit design as will be described below in more detail. Additional designs can also be created using variations of the stent manufacturing protocol.

Several methods may be used to manufacture the plugs or occluding devices disclosed herein. A septal defect or patent ductus arteriosus (PDA) plug can be fabricated by pouring the monomer solution into a glass tube (test tube) and photopolymerizing a solid cylinder of polymer. In cases of both the stent and plug fabrication, about 0.1 wt % photoinitiator may be used to obtain the best results of photocuring. This amount of photoinitiator may help prevent overheating of large batches of polymers and may also help prevent the monomers from boiling.

The first step in making the SMP stents and/or plugs is to create the polymer formulation itself. The specific polymers and methods of determining polymer formulations will be described in more detail below. Creating the polymer formulation is generally achieved by mixing two or more liquid monomers together and photopolymerizing them via a photoinitiator. This formulation is comprised of a linear monomer and a crosslinking monomer. The linear monomer is referred to as mono-functional because it only has one C═C double bond to react in the polymerization process, and thus can only grow as a linear chain. The crosslinking monomer is referred to as di-functional because it has two C═C double bonds and can form as an interconnect (crosslink) between the linear chains. In the present method of manufacturing SMP stents and plugs, tert-butyl acrylate (tBA) and poly(ethylene glycol) dimethacrylate (PEGDMA) may be used as respective mono- and di-functional monomers, as shown in FIG. 1.

The disclosed SMP devices have the ability to be polymerized by free radical initiation, which may be achieved by thermal or photoinitiators. The photoinitiator simply starts the polymerization reaction when exposed to heat or UV light. Without the photoinitiator, the tBA and PEGDMA will stay in their liquid monomer form. Both thermal and photoinitiation offer the ability to create bulk amounts of polymer or finely detailed geometries in a mould. In one aspect, photo-polymerization is employed because of its ability to completely polymerize the monomers within minutes. FIG. 11 illustrates exemplary glass transition curves of polymers cured for only a short amount of time. The curves are nearly identical after only 5 minutes.

In another embodiment, thermal-polymerization may be employed to slow the polymerization process, which usually takes place on the order of hours. Ultraviolet (UV) light first reacts with the photo-initiator to form free radicals, which go on to react with the functional C═C bonds to grow polymer chains and crosslinks. In one embodiment, the disclosed polymer formulation uses greater than about 10 wt % crosslinker.

In another embodiment, three or more monomers or homopolymers may be employed to achieve a greater range of achievable T_{g}'s, not just a linear line of possibilities. A shape-memory vascular defect device that is polymerized from 3 or more monomers may allow control of the glass transition temperature, percent crosslinking, and rubbery modulus in order to control the forces of shape recovery. In one aspect, the disclosed stents and plugs have high recovery force and rapid recovery through tailored crosslinking.

In some embodiments, heparin and other anti-thrombogenic, anti-proliferative, and anti-coagulant agents may be interspersed into the polymer matrix, or embedded into small biodegradable pockets within the stents or plugs for release with time. As mentioned above, SMP stents and plugs have the ability to infuse greater amounts of drugs into the polymer than other currently available polymer-coated metal stents.

Additionally, various radiopaque particles, bands or other markers may be infused into the polymers of the SMP stents and plugs to facilitate location of the SMP stents and plugs in vivo. The SMP stents and plugs having radiopaque particles therein may be easily located using x-ray, MRI and/or ultrasound imaging. In one embodiment the radiopaque particles may comprise gold powder, thin strips of gold foil, tungsten powder, gold-tantalum and iron-oxide, and iodipamide, and/or a radiopaque contrast medium. These radiopaque particles may also be used in diagnostic tests for the biliary system and are infused into the SMPs to enhance radio-opacity of the devices.

A transition temperature may be defined through a number of methods/measurements and different embodiments may use any of these different methods/measurements. For example, a transition temperature may be defined by a temperature of a material at the onset of a transition (Tonset), the midpoint of a transition, or the completion of a transition. As another example, a transition temperature may be defined by a temperature of a material at which there is a peak in the ratio of a real modulus and an imaginary modulus of a material (e.g., peak tan-delta), as is illustrated in FIG. 5. It should be noted that the method of measuring the transition temperature of a material may vary, as may the definition of steps taken to measure the transition temperature (e.g., there may be other definitions of tan-delta).

A transition temperature may be related to a number of processes or properties. For example, a transition temperature may relate to a transition from a stiff (e.g., glassy) behavior to a rubbery behavior of a material. As another example, a transition temperature may relate to a melting of soft segments of a material. A transition temperature may be represented by a glass transition temperature (T_{g}), a melting point, or another temperature related to a change in a process in a material or another property of a material.

In addition, molecular and/or microscopic processes, including those processes around a transition temperature, other SMP processes may be related to the macroscopic properties of the material. Indeed, one method of determining whether a molecular and/or microscopic process is occurring (or has occurred) is to monitor macroscopic processes or properties. Molecular and/or microscopic properties are commonly related to macroscopic properties, and macroscopic characteristics are commonly monitored as a substitute for monitoring molecular and/or microscopic properties.

From a macroscopic viewpoint, as embodied in a modulus-temperature graph, a polymer's shape memory effect may possess a glass transition region, a modulus-temperature plateau in the rubbery state. A polymer's shape memory effect may include, as embodied in stress-strain graph, a difference between the maximum achievable strain, εₘₐₓ, during deformation and permanent plastic strain after recovery, εₚ. The difference εₘₐₓ−εₚ may be considered the recoverable strain, recover, while the recovery ratio (or recovery percentage) may be considered ε_{recover}/εₘₐₓ.

Thermomechanic aspects of the polymer (T_{g} and % crosslinking) may be tailored during the polymer formulation of the SMP stents and plugs. Using a linear rule of mixtures (ROM), the glass transition of the polymer network can be determined by the fraction amounts of the individual components:

T_{final}=(wt %·T_{g})ₗᵢₙₑₐᵣ+(wt %·T_{g})_{crosslinker}

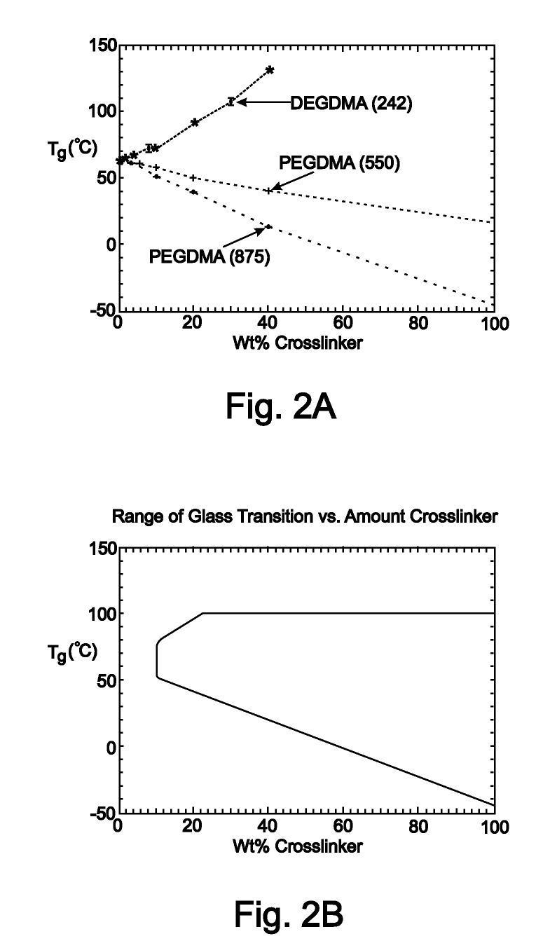

However, a two component mixture only allows a straight line of possibilities (see FIG. 2A). To further add control to the system, a third (or fourth) crosslinking monomer can be added to the system to help control the degree of crosslinking (see FIG. 2B). This new system follows the new ROM equation:

T_{final}=(wt %·T_{g})ₗᵢₙₑₐᵣ+[(wt %·T_{g})₁+(wt %·T_{g})₂]_{crosslinker}

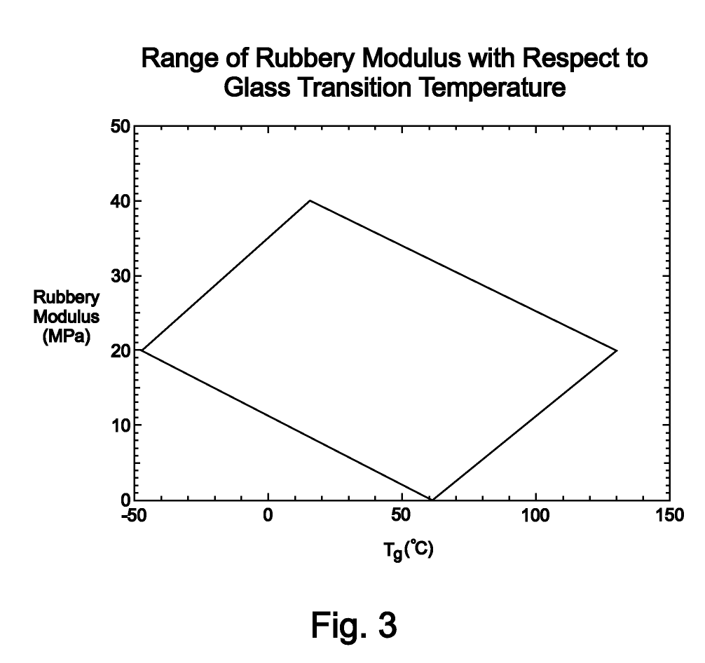

To tailor thermomechanical properties of the SMP stents and plugs, both T_{g} and crosslinking may be varied. Basic and clinical research indicates that most arteries have incremental Young's modulus between 0.5 MPa and 2 MPa, with increases beyond this due to severe diseases such as hypertension and atherosclerosis. In one embodiment, the disclosed stents and plugs may be designed to have modulus values between 1 and 50 MPa. In one embodiment, three or more homopolymers may be used to produce the resultant material and mechanical properties, which provides greater freedom to tailor properties than with materials that use only two homopolymers. For example, use of three homopolymers produces a more complex relationship between T_{g} and the rubbery modulus as shown in FIG. 3.

The modulus of elasticity, or rubbery modulus, for the stent and plug SMPs may be varied depending on application requirements. For example, in certain vascular applications the stents and/or plugs may need to deform along with natural vascular deformations of the blood vessel over the cardiac pumping cycle. This will require a low modulus of elasticity (˜1-5 MPa). For other applications including non-vascular applications, the stents may need to be exhibit greater rigidity to maintain an open blood vessel. This will require a larger modulus of elasticity (>20 MPa). Thus, one set of desired endpoints is the rubbery modulus of the stent. This can be controlled precisely by varying the crosslinking density of the polymer. Other stent properties may also be changed by varying the amount of crosslinking monomers, as will be described in further detail below. The optimal T_{g}, rubbery modulus and stent/plug geometry may vary depending on the application and particular vessel into which the stent and/or plug will be deployed. In one aspect, the optimal values lie within the following ranges: T_{g} of 45-55° C.; modulus between 1 to 50 MPa.

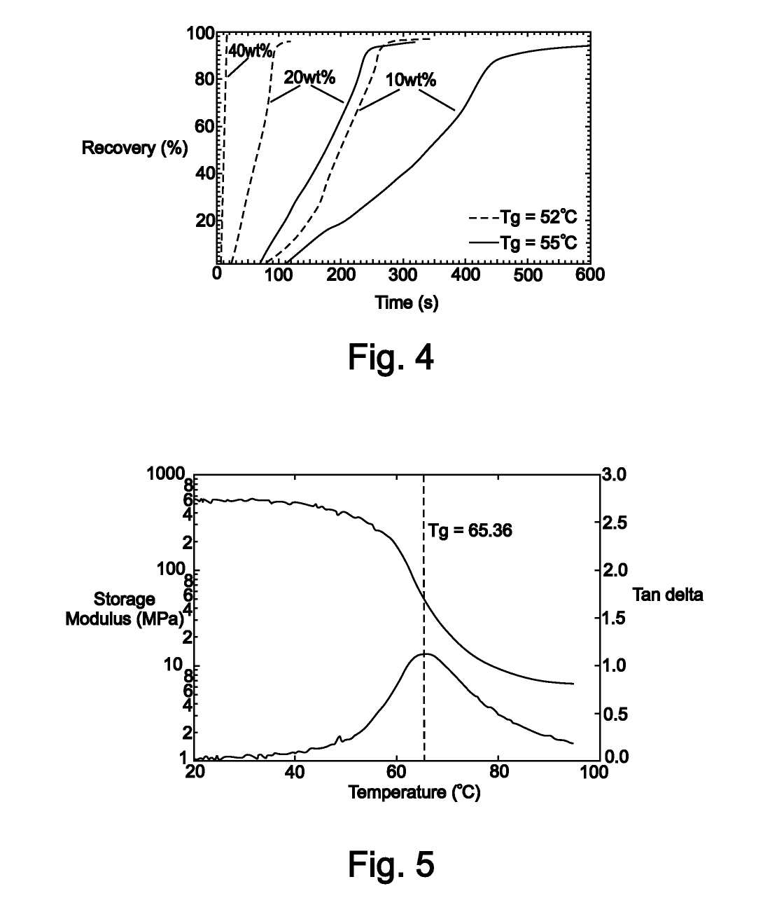

The deployment time of the SMP stents and plugs may need to be varied depending on application requirements. For example, certain vascular applications may require fast deployment to minimize the time that blood flow is blocked, whereas other applications including non-vascular applications may require gentler and slower deployment to minimize trauma to the vessel wall. Deployment time can be varied from 10 seconds to >550 seconds by varying crosslink density, as shown in FIG. 4. In one aspect, deployment time ranges from 60 seconds-600 seconds. Note that deployment time is affected more by % crosslink density than T_{g}. Deployment time of the stent can be controlled precisely by varying the crosslink density of the stent material. In one aspect, there is no requirement for external heating for stent deployment.

### Thermal Analysis

Thermomechanical characterization can be provided by dynamic mechanical analysis (DMA), dynamic scanning calorimetry (DSC), thermal gravimetric analysis (TGA), and standard tensile and compressive testing. DMA is the most useful of the tests because it quickly gives the most information of the polymer's thermomechanics.

In DMA, a sinusoidal force is applied to a polymer sample over a given temperature range. The machine will measure the response to this force and calculate the polymer's modulus, strain, and damping as a function of temperature. In a typical test, the polymer will go from a hard (glassy) state to a soft (rubbery) state. This is defined as a glass transition and is marked by a maximum peak in the damping curve (tan delta) (see FIG. 5).

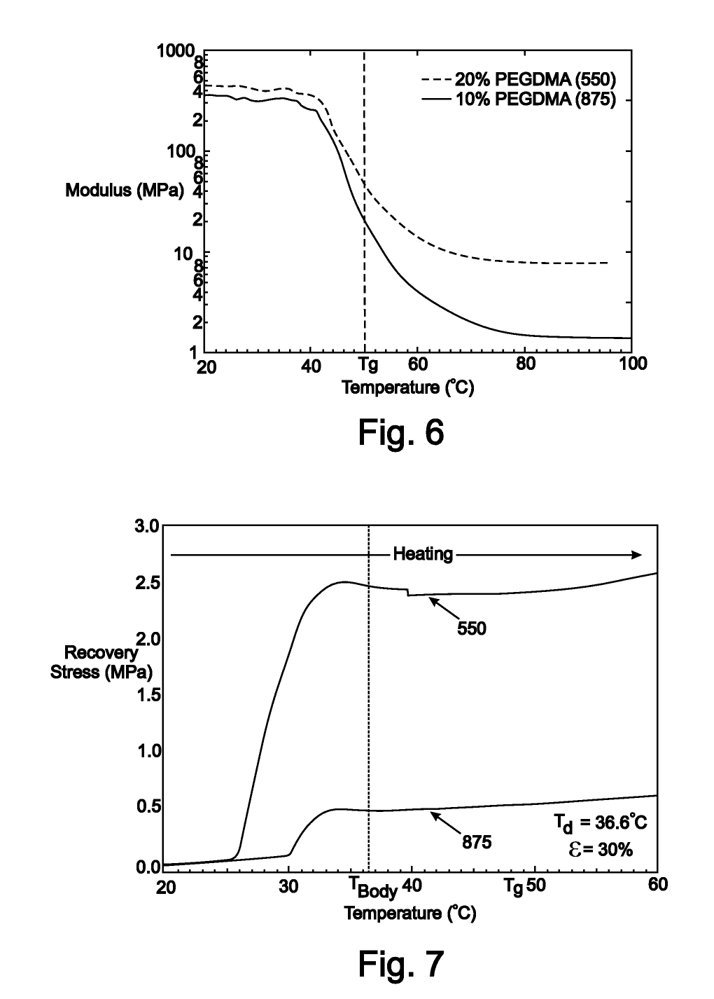

With respect to thermomechanical tailorablity, verification can be achieved by examining the T_{g} curves. Different amounts of crosslinking monomer can shift the T_{g} curve while also varying the modulus in the rubbery state. An increase in crosslinking (i.e. 10 wt %→20 wt %) will generally increase the rubbery modulus (1 MPa→10 MPa) (see FIG. 6).

Other methods of testing include DSC and compressive and tensile testing. DSC will measure transitions in a polymer by measuring the heat flow into the sample with reference to a standard. TGA measures the weight loss of the polymer as a function of temperature and shows thermal degradation at high temperatures. Compressive and tensile testing is done by a mechanical tester and measures the displacement of a sample as a function of force to give Young's modulus, yield, ultimate tensile strength, and strain to failure at a specific temperature.

### Recovery Force Measurements

Uniaxial, recovery-force measurements can be measured using a mechanical tester equipped with a thermal chamber. In this test, a tensile or compressive specimen is strained at a given temperature. The force to maintain this strain will decrease as the sample is cooled. Once the force to maintain this strain drops to zero, the sample is said to be in its stored or packaged state. The recovery force is then measured as the temperature inside the thermal chamber is steadily increased over time (see FIG. 7). Once the shape-memory effect is activated, a force to restore the strained sample will be recorded by the tester. Polymers with lower glass transition temperatures will activate at lower temperatures and polymers with a higher degree of crosslinking will have a higher recovery force.

Recovery force measurements on the actual stent or plug can be achieved using a micro-mechanical tester. A micro-mechanical tester is required because of the small-scale size of the stent and plug. This method is similar to the uniaxial method, however recovery force is measured from a more complex packaged state.

### Free Recovery Measurements

Free recovery of the stents or plugs may be measured via video by placing the packaged devices into a body temperature bath. Digital measurements can then be taken to measure the recovery as a function of time (see FIGS. 40A-40E). Typically, lower T_{g} and higher crosslinked polymers will recover faster at body temperature.

The stent should also allow re-expansion with full conformance to the vessel lumen after balloon expansion. Since increasing the wt % of the crosslinking monomer will increase stent stiffness, capability of the stent to re-expand will vary with wt % of the crosslinking monomer. Also, a slit-stent embodiment as further described below should have greater capacity to re-expand since it is not circumferentially constrained.

### Compliance Measurements

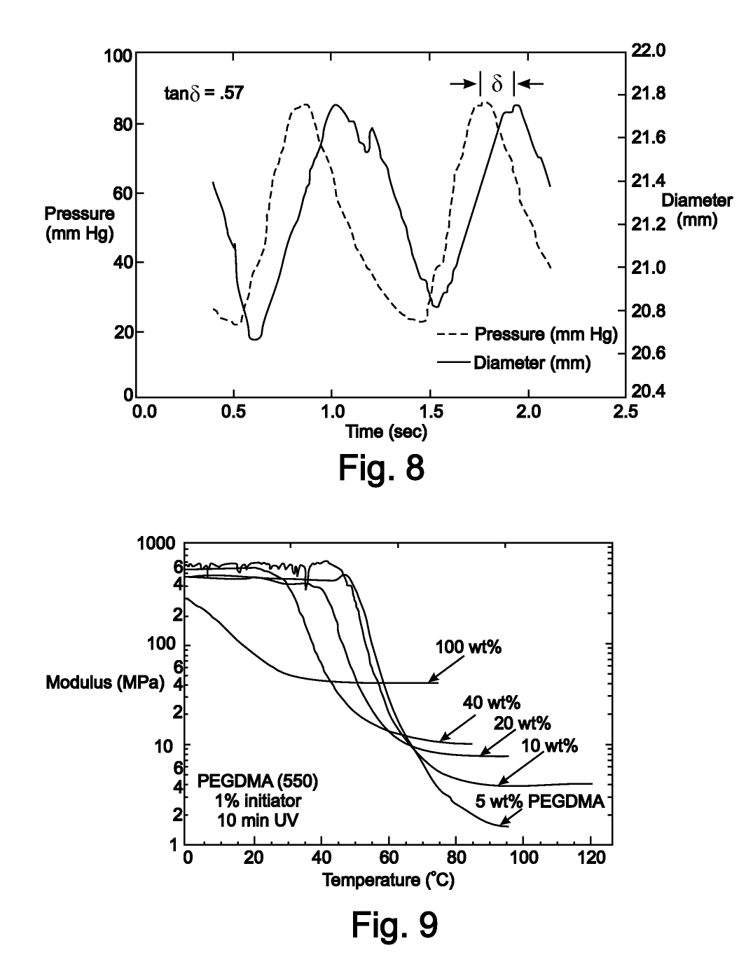

Compliance of the stents and plugs may be measured in-vitro or in-vivo using ultrasound imaging. By monitoring the pressure of the vessel, ultrasound imaging can correlate the diameter change of the stents and/or plugs with respect to pressure and time (see for example, FIG. 8). The compliance and distensibility of the stents and plugs can be calculated using different methods. For example, the following formulas can be used to calculate each:

$$\frac{{d}_{sys} - {d}_{dia}}{P} and \frac{{d}_{sys} - {d}_{dia}}{P \cdot {d}_{dia}}$$

(P=Pressure, d_{sys}=systolic diameter, d_{dia}-diastolic diameter).
   Other methods used to measure compliance include the pressure strain modulus (Eₚ) and dynamic compliance (C_{dyn}):

Eₚ(g/cm²)=ΔP×R_{d}/ΔR;

ΔP=pulse pressure; R_{d}=diastolic radius; ΔR=systolic minus diastolic radii)

(C_{dyn}(%/100 mmHg)=[ΔD/(ΔP×D_{d})]×10⁴
ΔD=systolic minus diastolic diameters; D_{d}=diastolic diameter).

In one aspect, SMP stent and plug design allows greater amounts of drugs to be infused into the polymer than with current polymer-coated metal stents or plugs. Depending on whether the device is meant to close a blood vessel or septal defect, the device can be tested in vitro using phantoms with water running through the phantom at a physiologically realistic flow rate and body temperature. Tubes (rigid and flexible) can be used to test devices to close blood vessels. An orifice phantom, mimicking cardiac and other septal defects, is used to test the septal defect closure system. Flow rate before and after deployment of the device can be used to gauge success of the device in sealing the defect. Video imaging of device deployment and seating can be used to gauge how well the device conforms to the inner lumen of the defect. T_{g} and % wt crosslink density can be varied based on application requirements to achieve modulus values between 1 and 50 MPa and deployment times between 1 and 500 seconds.

### EXAMPLES

Experimental work on exemplary SMP stents and plugs has been performed to demonstrate the feasibility and advantages of these polymers over currently used medical devices. The following examples are presented to demonstrate a SMP polymerization process, fabrication, characterization and testing of polymer materials for use in the SMP stents and plugs disclosed herein. These examples are provided for purposes of illustration only and are not intended to be limiting. All starting materials used in the below examples are commercially available.

A three-point flexural configuration was used for glass transition, free strain recovery, and stress recovery tests. In all tests, heating and cooling was typically performed at a constant rate of 5° C./min with data collection every 2 seconds. For example, in T_{g} tests, samples were cycled at a frequency of 1 Hz between minimum and maximum bending forces of 10 Mn and 90 Mn. The glass transition temperature (T_{g}) of the polymers varied over a range of 100° C. and is dependent on the molecular weight and concentration of the crosslinker. The polymers show 100% strain recovery up to maximum strains of approximately 80% at low and high deformation temperatures (T_{d}). Free strain recovery was determined to depend on the temperature during deformation; lower deformation temperatures (T_{d}<T_{g}) decreased the temperature required for free strain recovery. Constrained stress recovery shows a complex evolution as a function of temperature and also depends on T_{d}. Using variations of crosslinking density, nano reinforcement, fiber reinforcement, compression ratio (the amount of deformation), or layering, the SMP implant may withstand a range from 0.5 MPa to 20 MPa stress levels. The thermomechanical characterization was performed by dynamic mechanical analysis (DMA) on a Perkin Elmer Dynamic Mechanical Analyzer DMA-7.

### Example 1

### Control of Chemistry to Vary Mechanical Properties of Stents

### Protocol to Manufacture the Polymer Stents to Various Crosslinking Densities.

Tert-butyl acrylate (tBA), di(ethylene glycol) dimethacrylate (DEGDMA), poly(ethylene glycol) dimethacrylate (PEGDMA) with typical Mₙ=550 and Mₙ=875, and photoinitiator 2,2-dimethoxy-2-phenylacetephenone were ordered from Aldrich and used in their as received conditions without any further purification. Solutions were made by manually mixing the functionalized monomers at different mass fractions in a glass vial with 1 wt % photoinitiator. The solutions were injected into a thin-walled-tube mould to manufacture stents. The glass slides were separated with 1 mm spacers and coated with Rain-X, which acted as a non-reactive releasing agent. The thin-walled tube mould consisted of a Teflon rod (sizes: 3 mm-25 mm) sheathed with a slightly larger glass tube of diameter (3.2 mm-25.5 mm) (Allen Scientific) to create polymer tubes with a range of wall thickness values (100 μm to 500 μm) and outer diameters (3.2 mm-25.5 mm). Wall thickness increments were changed in 50 μm increments. Inner diameter increments were changed in 5 mm increments.

A UV-Lamp (Model B100AP; Black-Ray) was used to photopolymerize the solutions for 10 minutes at an intensity of 10 mW/cm². The mould is slowly but constantly rotated during photopolymerization. After processing of all materials, polymers were heat treated at 90° C. for 1 hour to ensure the complete conversion of monomers. The photoinitiator 2,2-dimethoxy-2-phenylacetephenone was used although a variety of other initiators can also be used. Results from cytotoxicity tests confirmed that use of the photoinitiator at about 0.01-1 wt % of the polymer system should not affect the biocompatibility of the polymer. Glass transition temperature (T_{g}) and rubbery modulus can be controlled by, for example, changing the weight percent (wt %) of the crosslinker, for example, PEGDMA (Mn 550) as shown in FIG. 9.

### Example 2

### Control of Geometry to Vary Mechanical Properties of Stents

### Protocol to Manufacture Solid Stents.

To manufacture solid stents, the glass tube was removed, leaving a long stent on the Teflon rod. Individual stents are then cut from the longer stent to a variety of lengths (0.5 cm-5 cm). The stent can be packaged as is, or with additional modifications, such as CNC machine modifications, which can be made based on application requirements, as detailed in Example 3 below.

### Example 3

### Protocol to Manufacture Porous Stents

To manufacture porous stents with different shapes and amounts of wall material removed, a laser-cutter was used to cut slots into the stent in a predefined pattern when the stent is still on the Teflon rod. Hole patterns in the size of circles, diamonds, and circumferentially or longitudinally oriented slits have been created. The amount of wall material removed is varied from 10% to 50%. The stents are then cut to preferred lengths and are ready for packaging.

### Example 4

### Deployment Time

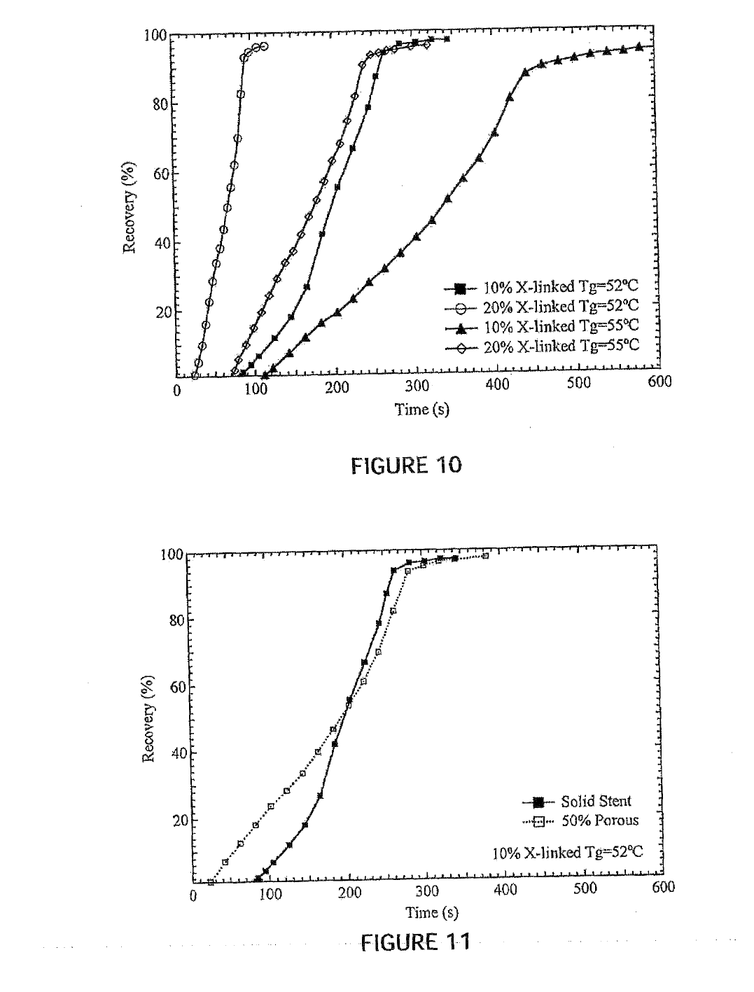

Deployment time of the stent can be controlled precisely by varying the crosslink density of the stent material. Crosslink density of the stent material can be varied as discussed above. In one example, 22 mm diameter stents were manufactured with different T_{g}'s and crosslink densities and tested for deployment time. The stents were manufactured using the method discussed above and packaged into an 18 F catheter at room temperature. The stent was then deployed into a body temperature water bath at 37° C. The stent was pushed out of the catheter and the time for the stent to recover to its cylindrical shape was measured. This was repeated for a porous stent, which had 50% of its wall material removed using a laser cutter as discussed above. FIGS. 10, 11, and 12 show experimental data of % recovery against time for different T_{g}'s and crosslink densities and for solid and 50% porous stents.

After packaging stents of various crosslink wt % into a catheter, the stents were deployed into a water bath at a temperature of 37 degrees Celsius. All stents deployed without requirement to heat the water temperature greater than 37 degrees Celsius. The rate of deployment varied as a function of crosslinking, Tg, and % porosity. Recovery times ranged from 10 seconds to >550 seconds by varying the T_{g} and crosslink density of the stent material. Smaller and greater recovery times can be obtained by varying these parameters even further.

### Example 5

### Dynamic Compliance

Compliance of the stent can be measured in-vitro or in-vivo using ultrasound imaging. By monitoring the pressure of the vessel, ultrasound imaging can correlate the diameter change of the stent with respect to pressure and time. The compliance and distensibility of the stent can be calculated using different methods. For example, dynamic compliance (C_{dyn}) has been used, and is calculated by:

C_{dyn}(%/100 mmHg)=[ΔD/(ΔP×D_{d})]×10⁴
ΔD=systolic minus diastolic diameters; D_{d}=diastolic diameter).

In this experiment, dynamic compliance ranges from about 5% change/100 mm Hg-50% change/100 mm Hg.

### Example 6

### Stent Deployment

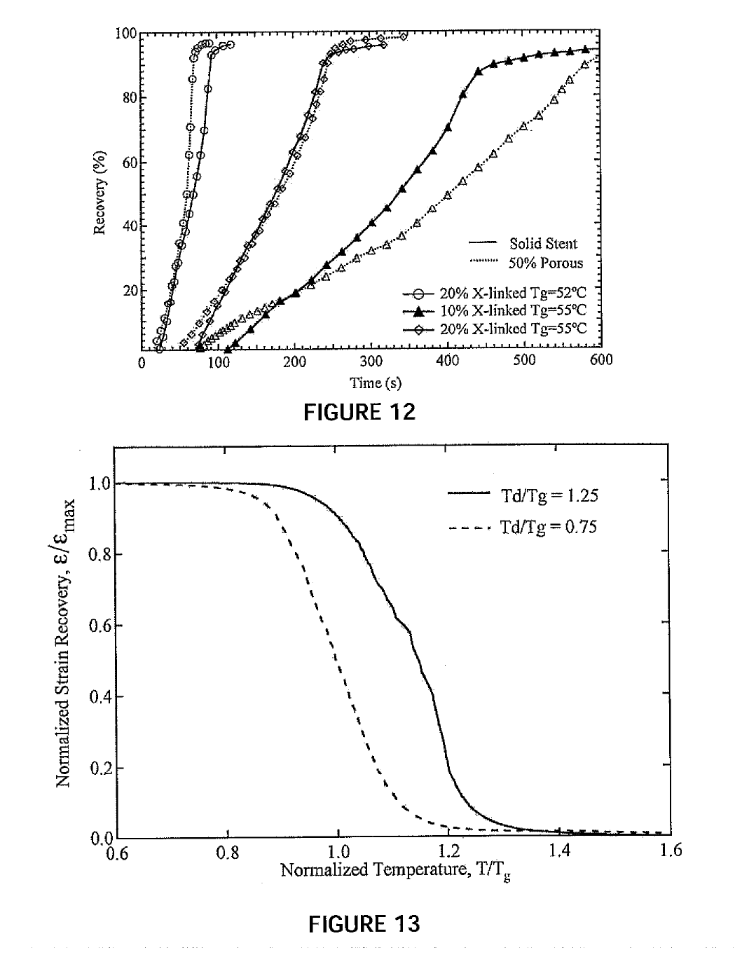

After packaging stents of various crosslink wt % into a catheter, the stents were deployed into a water bath at a temperature of 37 degrees Celsius. All stents deployed without requirement to heat the water temperature greater than 37 degrees Celsius. The rate of deployment varied as a function of crosslinking, as shown in FIG. 12. In the figure, % porosity was another variable tested. An in vitro recovery of a shape memory polymer stent at body temperature after deployment via catheter is shown in FIG. 12.

### Example 7

### Ability to Store at Room Temperature

The ability to store at room temperature depends on the glass transition temperature (T_{g}) as well as packing temperature (T_{d}). FIG. 13 shows a free strain recovery graph of a bent sample with respect to its T_{g}. This figure shows that lower packaging temperatures initiate recovery at 0.7 T_{g} whereas higher packaging temperatures initiate recovery 0.9 T_{g}. Therefore, a polymer with a T_{g}=50° C. will activate at 35° C. when packaged at a low temperature. At room temperature, 25° C., the polymer will remain stable and unactivated.

In general, stents were manufactured, packaged and stored in a container with phosphate-buffered saline (PBS) at room temperature. These containers were then opened after various time periods and tested using thermomechanical characterization tests for thermomechanical variations in function. Stents were stored up to 6 months and exhibited no changes in thermomechanical characteristics.

### Example 8

### Infusion of Various Radiopaque Particles into the Polymer to Facilitate Location of the Devices Via X-Ray, MRI and/or Ultrasound Imaging

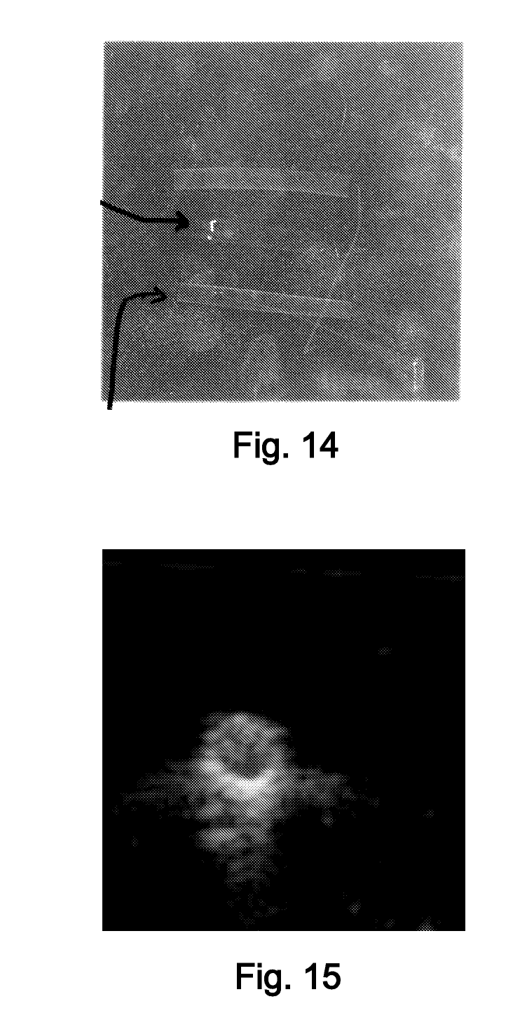

Gold powder, thin strips of gold foil, tungsten powder, and iodipamide have all been infused into the stent material to enhance radio-opacity. FIG. 14 shows representative X-ray images of stents with 4% by weight iodipamide and gold foil. FIG. 15 shows a cross-sectional, B-mode ultrasound image of the stent within a water bath taken using 7.5 MHz imaging frequency.

### Example 9

### Significant and Tailorable Crush Recovery Properties

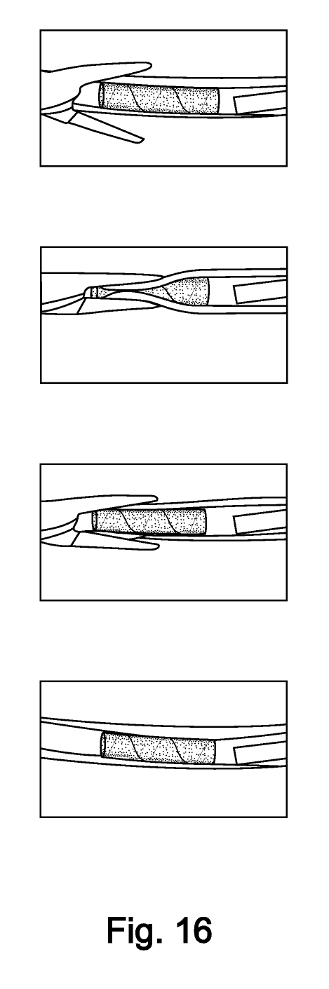

Stents are manufactured with varying crosslinking wt % (10%-40%). Stents are then deployed into a mock compliant artery (silicone rubber tube) with water at body temperature (37° C.) circulating through. The stent is then crushed using pliers. The stent recovery behavior is captured using a video camera. The time recovery of the stent after crushing is analyzed and recovery time after crushing is plotted against crosslink wt %. FIG. 16 illustrates sequential images of a stent recovering from a crush recovery experiment.

### Example 10

### Ability to Exert Stress Upon Recovery

Uniaxial recovery-force measurements are measured using a mechanical tester equipped with a thermal chamber. A tensile or compressive specimen is strained at a given temperature. The force to maintain this strain will decrease as the sample is cooled. Once the force to maintain this strain drops to zero, the sample is said to be in its stored or packaged state. The recovery force is then measured as the temperature inside the thermal chamber is steadily increased over time (FIG. 7). Once the shape-memory effect is activated, a force to restore the strained sample is recorded. Generally, polymers with lower glass transition temperatures activate at lower temperatures and polymers with a higher degree of crosslinking will have a higher recovery force. Recovery force measurements on the actual stent or plug are achieved using a micro-mechanical tester. A micro-mechanical tester is required because of the small-scale size of the stent and plug. This method is similar to the uniaxial method, however, recovery force is measured from a more complex packaged state. In general, recovery forces range from 0.2 MPa to 5.0 MPa based on crosslink wt % configuration as shown in FIG. 7.

### Example 11

### Ability for the Stent to Grow with the Vessel

Various methods are used to create stents that can grow as the vessel into which the stent has been placed also grows. For example, a 5 mm diameter solid stent was cut longitudinally prior to packaging. This stent was then deployed into glass tubes ranging in internal diameters from 4 mm to 8 mm with water flowing through at body temperature (37° C.). The stent was able to fit the internal lumen of all tubes upon delivery with no slipping, sliding or buckling. Experiments to test this property include manufacturing stents with both solid and slit configurations, deploying these into tubes of varying sizes with water flow, and examining how well the stent conforms to the internal lumen of the tube wall for different tube and stent sizes. Evidence of non-conformance will include stent buckling, slipping, sliding or other shape deformation, which prevents the stent from fully conforming to the vessel wall.

### Example 12

### Ability to Re-Expand the Stent Using Balloon Angioplasty

Stents at varying crosslink wt % (10%-40%) and geometry (solid and slit) are packaged and deployed into a mock artery (silicone rubber tube) with water flowing through at body temperature (37° C.). Stenosis of the artery is simulated by temporarily pushing down on one surface of the stent and mock artery. A conventional balloon catheter is deployed into the lumen and the balloon is expanded at the location of the stenosis. A video recording is employed to determine whether the stent conforms to the vessel wall after deployment. The stent is further examined for surface damage using visual, microscopic and electron microscopy inspection after removal from the mock artery.

### Example 13

### Protocol to Manufacture and Test Solid Polymer Plugs

Chemicals and processes to manufacture the SMP plugs follow the stent protocols. All variables that can be changed for the stent (% wt crosslinking, T_{g}, etc.) can also be varied to the same extent for the polymer plugs. After mixing the polymers, the solutions were poured into molds of different shapes and UV cured to create the plugs. Molds were removed, resulting in plugs of various shapes as shown in FIG. 50. The plugs may then be packaged for delivery.

In addition to solid plugs, a variety of other designs were created for the vascular defect closure system. FIG. 51 shows additional designs realized using the acrylate-based shape memory polymer. These designs were tested for deployment rate; data are shown in Table 1.

TABLE 1
Deployment times for vessel occlusion
device designs shown in FIG. 51.

| | Diameters (mm) | | | |
|---|---|---|---|---|
| Design | Ex- | Com- | Deployment | |
| # | panded | pressed | Speed | Deployment Description |
| 1(base) | 11 | N/A | N/A | N/A |
| 2 | 11 | 6.4 | <1 sec | All legs outward evenly |
| 3 | 11 | 7 | <1 sec | Evenly uncoiling |
| 4 | 11 | 7.8 | <1 sec | One leg at a time |
| 5 | 11 | 6 | <1 sec | All legs outward evenly |

### Example 14

### Packaging the SMP Vascular Defect Closure System

The device is heated (to 40-50° C.) and stretched to reduce the cross-section of the device and enable packaging into a small catheter. The device is then cooled to room temperature. Once the device is completely cooled, it is ready to be inserted into the delivery catheter for deployment.

### Polymers Used to Fabricate SMPs

The first step in making a SMP stent or plug device is to create the polymer formulation itself. Exemplary polymers used for forming SMP stent and plug devices are disclosed herein. In certain embodiments, the SMP polymer segments may be natural or synthetic. The polymer segments can be biodegradable or non-biodegradable. In general, biodegradable materials degrade by hydrolysis, by exposure to water or enzymes under physiological conditions, by surface erosion, by bulk erosion, or a combination thereof. Non-biodegradable polymers used for medical applications preferably do not include aromatic groups, other than those present in naturally occurring amino acids. The SMP stents and plugs disclosed herein may be biodegradable or nonbiodegradable.

The polymers are selected based on the desired glass transition temperature(s) (if at least one segment is amorphous) or the melting point(s) (if at least one segment is crystalline), which in turn is based on the desired applications, taking into consideration the environment of use or final application. Representative natural polymer blocks or polymers include proteins such as zein, modified zein, casein, gelatin, gluten, serum albumin, and collagen, and polysaccharides such as alginate, celluloses, dextrans, pullulane, and polyhyaluronic acid, as well as chitin, poly(3-hydroxyalkanoate)s, especially poly(.beta.-hydroxybutyrate), poly(3-hydroxyoctanoate) and poly(3-hydroxyfatty acids). Representative natural biodegradable polymer blocks or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Representative synthetic polymer blocks or polymers include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof. Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt. These are collectively referred to herein as “celluloses”.

Representative synthetic degradable polymer segments include polyhydroxy acids, such as polylactides, polyglycolides and copolymers thereof, poly(ethylene terephthalate); polyanhydrides, poly(hydroxybutyric acid); poly(hydroxyvaleric acid); poly[lactide-co-(.epsilon.-caprolactone)]; poly[glycolide-co-(.epsilon.-caprolactone)]; polycarbonates, poly(pseudo amino acids); poly(amino acids); poly(hydroxyalkanoate)s; polyanhydrides; polyortho esters; and blends and copolymers thereof. Polymers containing labile bonds, such as polyanhydrides and polyesters, are well known for their hydrolytic reactivity. Their hydrolytic degradation rates can generally be altered by simple changes in the polymer backbone and their sequence structure.

Examples of non-biodegradable synthetic polymer segments include ethylene vinyl acetate, poly(meth)acrylic acid, polyamides, polyethylene, polypropylene, polystyrene, polyvinyl chloride, polyvinylphenol, and copolymers and mixtures thereof.

The polymers disclosed herein may be obtained from commercial sources such as Sigma Chemical Co., St. Louis, Mo.; Polysciences, Warrenton, Pa.; Aldrich Chemical Co., Milwaukee, Wis.; Fluka, Ronkonkoma, N.Y.; and BioRad, Richmond, Calif. Alternately, the polymers can be synthesized from monomers obtained from commercial sources, using standard techniques.

In one embodiment, the SMPs may be photopolymerized from tert-butyl acrylate (tBA) di-functional monomer with polyethylene glycol dimethacrylate (PEGDMA) tetra-functional monomer acting as a crosslinker. A di-functional monomer may be any compound having a discrete chemical formula further comprising an acrylate functional group that will form linear chains. A tetra-functional monomer may be any compound comprising two acrylate, or two methacrylate groups. A crosslinker may be any compound comprising two or more acrylate or methacrylate functional groups. Also, ethyleneglycol, diethyleneglycol, and triethyleneglycol-based acrylates are forms of polyethyleneglycol-based acrylates with only one, two, or three repeat units.

In another embodiment, the SMP stents and plugs may be photopolymerized from three or more monomers and/or homopolymers to achieve a range of desired thermochemical properties. An SMP device formed from three or more monomers and/or homopolymers may achieve a range of rubbery modulus to glass transition temperature (see FIG. 3), rather than strictly a linear relationship between these two thermomechanical properties. For example, a combination of tert-butyl acrylate (tBA), polyethylene glycol dimethacrylate (PEGDMA), and diethyleneglycol dimethacrylate (DEGDMA), may be employed in SMP photopolymerization.

SMP devices can be designed to provide various mechanical properties. In one embodiment, the amount of crosslinker used in SMP polymerization is greater than about 10%. The SMP stents and plugs may be designed to have modulus values between 1 and 50 MPa. The deployment time may be varied from about 10 seconds to about 550 seconds. Further, the SMP stents and plugs may have solid or porous geometry. The SMP stents and plugs may also contain an internal scaffold that flexes with vessel pulsation.

As mentioned above, the SMP stents and plugs may be formed of a photo-initiated network comprising of tert-butyl acrylate (tBA), polyethyleneglycol dimethacrylate (PEGDMA), and 2,2-dimethoxy-2-phenylacetephenone as a photo-initiator, as shown in FIG. 1. In one embodiment, by controlling the amount of crosslinking PEGDMA, the glass transition temperature (T_{g}) was tailored to from about 45° C. to about 55° C., which makes the polymer optimal for shape recovery at body temperature. Other polymerization techniques, such as thermal radical initiation, can be used for polymer fabrication.

The SMP stents and plugs may be photopolymerized from several different monomers and/or homopolymers to achieve a range of desired thermomechanical properties. An SMP formed from three or more monomers and/or homopolymers may achieve a range of rubbery modulus to glass transition temperature, rather than a strictly linear relationship between these two thermomechanical properties. For example, tert-butyl acrylate (tBA) may be substituted by 2-hydroxyethyl methacrylate or methyl methylacrylate to create either more hydrophilic or stronger networks, if desired. Additionally, if a hydrophilic monomer such as 2-hydroxyethyl methacrylate is substituted for tert-butyl acrylate (tBA), the SMP may have the ability to further swell post-implantation through hydrogel mechanisms. The swelling post-implantation may provide for further expansion of the SMP plugs, which allows the SMP to adapt to changes in vessel size after implantation and keep the SMP plug in place even if the vessel changes or adjusts in size, shape, or curvature.

In some embodiments, hydrogels may be incorporated into the SMP plugs and formed from polyethylene glycol, polyethylene oxide, polyvinyl alcohol, polyvinyl pyrrolidone, polyacrylates, poly(ethylene terephthalate), poly(vinyl acetate), and copolymers and blends thereof. It may be desirable to use a hydrogel, such as 2-hydroxyethyl methacrylate (2-HEMA; as shown in FIG. 1) in place of, or in conjunction with, the tert-butyl acrylate (tBA). The incorporation of these hydrogel materials may cause the polymer material to soften and swell as it absorbs water over time. The incorporation of these hydrogel materials may also increase the thickness of the SMP occluding devices (e.g., plugs), helping to more completely block the vessel or more effectively achieve septal occlusion.

In some implementations it may be desirable to use 2-hydroxyethyl methacrylate (2-HEMA) in place of, or in conjunction with, tert-butyl acrylate. 2-hydroxyethyl methacrylate (2-HEMA) is illustrated in FIG. 1. 2-HEMA, also known as ethylene glycol methacrylate, forms a highly hydrophilic polymer and is most known for its use in contact lenses. 2-HEMA has a similar structure to the crosslinking monomer and has a similar glass transition temperature (˜70° C.), as compared to tert-butyl acrylate (˜60° C.). By incorporating 2-HEMA into the polymer synthesis, it is possible to create SMPs with a higher affinity toward water, which will cause the polymer to soften and swell by absorbing water over time. The degree by which the polymer will swell will be controlled by the amount of 2-HEMA and the amount of crosslinking within the matrix.

The combination of SMPs with the 2-HEMA polymer provides unique structural and functional advantages. For example, the SMPs may provide an ability to expand the SMP plugs significantly (shown in FIGS. 53 and 54) from a compacted state in the delivery catheter, and thereby allow for immediate effectiveness as an occluding device. The use of the 2-HEMA polymer provides a water absorption aspect to allow further expansion over time to “lock” the SMP plugs in place to ensure the SMP plug will be permanently implanted. The ability to swell by water absorption also provides the ability to fine-tune how well the SMP plug conforms to the vessel lumen, which may be particularly important for complex anatomy.

In addition to the above described materials, the SMP stents and plugs may be manufactured using a combination of SMP and non-SMP materials. The addition of the non-SMP materials may help to increase mechanical strength of the SMP stents and plugs. In one example, different weight fractions of reinforcing fibers (non-SMP materials) may be added to enhance durability and resistance to tearing. This mixed polymer may be formed by selecting the appropriate glass transition temperature and appropriate percentage of crosslinking monomer and then blending in the typical fashion. After blending, an appropriate amount of photoactivated initiator may be added. The mixture may be agitated until the initiator is completely dissolved. Once the initiator has been dissolved the mixture is ready for polymerization and may be set aside. An appropriately shaped mould may be made, typically out of glass slides held 1-2 mm apart by a non-reactive rubber spacer. Once the mould is prepared, the reinforcing fibers (i.e., non-SMP materials) may be added to the mould, and it is sealed closed. The reinforcing fibers used in this process may be short, for example averaging 150μ in length and 7-10μ in diameter. However, it is contemplated that the length of the fibers may be altered. The prepared monomer solution may then be injected into the mould. The entire mould may then be vigorously agitated both before and during polymerization to ensure the reinforcing fibers will be evenly distributed in the final product.

The drawings attached hereto are intended to further illustrate and exemplify the SMP stents and plugs described herein. These exemplary drawings are for purposes of illustration only and the dimensions, sizes and shapes reflected in the drawings attached hereto may vary. These SMP stents and plugs may be formed in a variety of sizes and shapes and any sizes, rates, times, or measurements given above are exemplary in nature only and are not meant to be limiting.

The above description, examples and data provide a complete description of the structure and use of example embodiments of the SMP stents and plugs. Although various embodiments of the invention have been described above with a certain degree of particularity, or with reference to one or more individual embodiments, those skilled in the art could make numerous alterations to the disclosed embodiments without departing from the spirit or scope of these SMP stents and plugs. It is intended that all matter contained in the above description and shown in the accompanying drawings shall be interpreted as illustrative only of particular embodiments and not limiting. Changes in detail or structure may be made without departing from the basic elements of these SMP stents and plugs.

What is claimed is:

1. A shape memory polymer (SMP) device for in vivo medical applications, formed from a first monomer and a second crosslinking monomer, wherein a first weight percentage of the first monomer and a second weight percentage of the second monomer are selected to reach one or more predetermined thermomechanical properties of the SMP device to optimize post-implantation memory shape properties of the SMP device.

2. The SMP device of claim 1, wherein the first monomer comprises tert-butyl acrylate (tBA) and wherein the second crosslinking monomer comprises polyethyleneglycol dimethacrylate (PEGDMA).

3. The SMP device of claim 1, further comprising at least one additional monomer.

4. The SMP device of claim 1, further comprising at least one additional homopolymer.

5. The SMP device of claim 45, wherein the glass transition temperature (T_{g}) ranges from about 45° C. to about 55° C.

6. The SMP device of claim 45, wherein the rubbery modulus ranges from about 1 MPa to about 50 MPa.

7. The SMP device of claim 1, wherein the device comprises a stent.

8. The SMP device of claim 7, wherein the stent is at least partially perforated.

9. The SMP device of claim 7, wherein the stent is fenestrated.

10. The SMP device of claim 7, wherein the stent comprises an outer surface defining a longitudinal slit and the stent has a post-implantation memory shape variable between a tube with overlapping edges to a C-shape with separated edges.

11. The SMP device of claim 7, wherein the stent comprises and outer surface defining a number of circumferential slits and the stent has a substantially cylindrical post-implantation memory shape.

12. The SMP device of claim 7, wherein the stent has a coiled post-implantation memory shape.

13. The SMP device of claim 7, wherein the stent has a substantially cylindrical post-implantation memory shape.

14. The SMP device of claim 1, wherein the device comprises an anatomical lumen occlusion device for either permanent occlusion, temporary occlusion, or partial occlusion to providing liquid flow control within the lumen.

15. The SMP device of claim 14, wherein the lumen occlusion device has a bulbous post-implantation memory shape.

16. The SMP device of claim 14, wherein the lumen occlusion device has a coiled post-implantation memory shape.

17. The SMP device of claim 14, wherein the lumen occlusion device is infused with at least one hydrogel material to increase swelling of the lumen occlusion device upon absorbing fluid.

18. The SMP device of claim 14, wherein the lumen occlusion device further comprises a securing mechanism to engage with a lumen wall to inhibit movement of the lumen occlusion device with respect to the lumen wall.

19. (canceled)

20. The SMP device of claim 14, wherein the lumen occlusion device further comprises a guidewire to control and guide the lumen occlusion device into a proper position during implantation.

21. The SMP device of claim 14, wherein the lumen occlusion device further comprises an elongated tail portion to control and guide the lumen occlusion device into a proper position during implantation.

22. The SMP device of claim 1, wherein the SMP device is substantially uniformly infused with at least one therapeutic medication.

23. The SMP device of claim 1, further comprising a reservoir portion to hold at least one therapeutic medication.

24. The SMP device of claim 1, wherein the SMP device is capable of assuming a memory shape at physiological temperatures.

25. The SMP device of claim 1, wherein the SMP device is compacted for delivery and rebounds back to an original configuration post implantation.

26. The SMP device of claim 1, wherein the SMP device recovers its original shape when heated to body temperature.

27. The SMP device of claim 1, wherein the SMP device retains its compacted shape when kept at or below about 25° C.

28. The SMP device of claim 1, further comprising a radiopaque material.

29. The SMP device of claim 1, wherein the first weight percentage the second weight percentage are selected by performing an iterative function to achieve a desired range of the thermomechanical properties.

30. The SMP device of claim 1, wherein the thermomechanical properties comprise one or more of the following:
a predeformation temperature (T_{d}),
a storage temperature (Tₛ),
a recovery temperature (Tᵣ), and/or
a deployment time.

31. The SMP device of claim 1, wherein the SMP device is formed to a size slightly larger than a target anatomical lumen size, to conform to and be stable in an applied position and to be capable of adapting to physiological pressure movement, and changes in an anatomical lumen size, while maintaining conformance and the applied position.

32. A method of forming a shape memory polymer (SMP) device for in vivo medical applications, comprising:
selecting a first monomer and a second crosslinking monomer, wherein the weight percentage of the first monomer and the weight percentage of the second monomer are selected to reach one or more predetermined thermomechanical properties of the SMP device to optimize post-implantation memory shape properties of the SMP device;
preparing a polymer formulation by combining the first and second monomers with a photoinitiator;
introducing the combined first and second monomers into a mould formed in a desired post-implantation shape of the SMP device;
photopolymerizing the polymer formulation in the mould to form the SMP device in the desired post-implantation shape; and
removing the mould to expose the SMP device formed in the desired post-implantation shape with optimal post-implantation memory shape properties.

33. The method of claim 32, wherein adding a photoinitiator comprises adding 2,2-dimethoxy-2-phenylacetophenone.

34. The method of claim 32, wherein preparing the polymer formulation further comprises adding a hydrogel material to the polymer formulation to increase the post-implantation size of the SMP device upon absorbing fluid.

35. The method of claim 34, wherein adding the hydrogel material to the polymer formulation comprises adding 2-hydroxyethyl methacrylate to the polymer formulation.

36. The method of claim 32, wherein preparing the polymer formulation further comprises adding at least one therapeutic medication to the polymer formulation for later release.

37. The method of claim 32, wherein preparing the polymer formulation further comprises adding a radiopaque material to enhance detection of the SMP device.

38. (canceled)

39. The method of claim 32, further comprising adding a reservoir to the SMP device before or during photopolymerizing the polymer formulation, wherein the reservoir is capable of holding therapeutic drugs.

40. The method of claim 32, wherein the selecting operations further comprise performing an iterative function to adjust the first weight percentages and the second weight percentage to achieve a desired range of the thermomechanical properties.

41. The method of claim 32, wherein the mould is selected to be slightly larger than a target anatomical lumen size to form the SMP device capable of conforming to and stable in an applied position, and capable of adapting to physiological pressure, movement, and changes in an anatomical lumen, while maintaining conformance and the applied position.

42. The method of claim 32, further comprising deforming the SMP device by
cooling the SMP device; and
compacting the SMP device before implantation.

43. The method of claim 32, wherein the first monomer comprises tert-butyl acrylate (tBA) and wherein the second crosslinking monomer comprises polyethyleneglycol dimethacrylate (PEGDMA).

44. A shape memory polymer (SMP) stent for in vivo applications, formed from a first monomer and a second crosslinking monomer, wherein a weight percentage of the second crosslinking monomer is selected by performing an iterative function to reach one or more predetermined thermomechanical properties of the SMP stent to optimize post-implantation memory shape properties of the SMP stent.

45. The SMP stent of claim 44, wherein the thermomechanical properties of the SMP stent comprise a predetermined glass transition temperature (T_{g}) and a predetermined rubbery modulus.

46. The SMP stent of claim 44, wherein the second crosslinking monomer comprises polyethyleneglycol dimethacrylate (PEGDMA).

47. The SMP device of claim 44, wherein the thermomechanical properties comprise one or more of the following:
a predeformation temperature (T_{d}),
a storage temperature (Tₛ),
a recovery temperature (Tᵣ), and/or
a deployment time.

48. A shape memory polymer (SMP) vascular occlusion device for in vivo applications, formed from a first monomer and a second crosslinking monomer, wherein a weight percentage of the second crosslinking monomer is selected by performing an iterative function to reach one or more predetermined thermomechanical properties of the SMP vascular occlusion device to optimize post-implantation memory shape properties of the SMP vascular occlusion device.

49. The SMP vascular occlusion device of claim 48, wherein the thermomechanical properties of the SMP vascular occlusion device comprise a predetermined glass transition temperature (T_{g}) and a predetermined rubbery modulus.

50. The SMP vascular occlusion device of claim 48, wherein the second crosslinking monomer comprises polyethyleneglycol dimethacrylate
(PEGDMA).

51. The SMP vascular occlusion device of claim 48, wherein the thermomechanical properties comprises one or more of the following:
a predeformation temperature (T_{d}),
a storage temperature (Tₛ),
a recovery temperature (Tᵣ), and/or
a deployment time.

52. A shape memory polymer (SMP) occlusion device for in vivo applications having a substantially bulbous post-implantation memory shape.

53. The SMP occlusion device of claim 52, further comprising a hydrogel material to increase the post-implantation size of the SMP occlusion device upon absorbing fluid.

54. The SMP occlusion device of claim 53, wherein the hydrogel material comprises 2-hydroxyethyl methacrylate.

55. The SMP occlusion device of claim 52, further comprising a securing mechanism to engage with a lumen wall to inhibit movement of the SMP occlusion device with respect to the lumen wall.

56. The SMP occlusion device of claim 52, further comprising a guidewire to control and guide the SMP occlusion device into a proper position during implantation.

57. A shape memory polymer (SMP) occlusion device for in vivo applications having a substantially coiled post-implantation memory shape.

58. The SMP occlusion device of claim 57, further comprising a hydrogel material to increase the post-implantation size of the SMP occlusion device upon absorbing fluid.

59. The SMP occlusion device of claim 58, wherein the hydrogel material comprises 2-hydroxyethyl methacrylate.

60. The SMP occlusion device of claim 57, further comprising a securing mechanism to engage with a lumen wall to inhibit movement of the SMP occlusion device with respect to the lumen wall.

61. The SMP occlusion device of claim 57, further comprising a guidewire to control and guide the SMP occlusion device into proper position during implantation.

62. A shape memory polymer (SMP) stent device for in vivo applications having a substantially cylindrical post-implantation memory shape with an outer surface defining a longitudinal slit, wherein a post-implantation memory shape of the SMP stent device ranges from a tube with overlapping edges along the longitudinal slit to a C-shape with separated edges.

63. A shape memory polymer (SMP) stent device for in vivo applications having a substantially cylindrically coiled post-implantation memory shape.

64. The SMP device of claim 1, wherein a recovery stress of the SMP device post implantation within an anatomical lumen generates forces that push out on tissue to create a central cavity.

65. The SMP device of claim 1, wherein the SMP device further comprises a non-SMP material to enhance a specific mechanical property or achieve a specific function of the SMP device.

66. The SMP device of claim 1, wherein the thermomechanical properties of the stent comprise a predetermined glass transition temperature (T_{g}) and a predetermined rubbery modulus.

67. The SMP device of claim 17, wherein the hydrogel material comprises 2-hydroxyethyl methacrylate.

68. The method of claim 32, wherein the thermomechanical properties of the SMP device comprise a predetermined glass transition temperature (T_{g}) and a predetermined rubbery modulus.
